(19) United States
(12) Patent Application Publication
(10) Pub. No.: US 2025/0097569 A1
(43) Pub. Date: Mar. 20, 2025

(54) INTERACTIVE MULTIMEDIA COLLABORATION PLATFORM WITH REMOTE-CONTROLLED CAMERA AND ANNOTATION

(71) Applicant: Datasya Ltd., Petah Tikva (IL)

(72) Inventors: Moshe Bartov, Petah Tikva (IL); Gad Terliuc, Ra'anana (IL)

(21) Appl. No.: 18/965,792

(22) Filed: Dec. 2, 2024

Related U.S. Application Data
(63) Continuation of application No. PCT/US2023/024197, filed on Jun. 1, 2023.

(51) Int. Cl. H04N 23/60; G06F 3/01; G06F 3/0346; G06F 21/62; G11B 27/10; H04N 5/76; H04N 23/61; H04N 23/63; H04N 23/66

(52) U.S. Cl. CPC H04N 23/64; G06F 3/016; G06F 3/0346; G06F 21/6254; H04N 5/76; H04N 23/61; H04N 23/634; H04N 23/66; G11B 27/10

(57) ABSTRACT

Systems and methods are provided for facilitating collaboration using visual content or multimedia content with a visual component, and alternative presentation and documentation modes of exchanged discussions, visual content and multimedia content.

### REFERENCE TO RELATED APPLICATIONS

The present application is a continuation of International Application No. PCT/US2023/024197, filed Jun. 1, 2023 and titled “Interactive Multimedia Collaboration Platform With Remote-Controlled Camera and Annotation”, which claims priority to U.S. Provisional Patent Application No. 63/365,837, field Jun. 3, 2022 and titled “Remote Controlled Camera System for Health Care Professionals,” and to U.S. Provisional Patent Application No. 63/384,380, filed Nov. 18, 2022 and titled “Camera Stabilization System for Health Care Professionals,” the disclosures of each of which are hereby incorporated by reference and made part of this specification.

### BACKGROUND

### Field

This disclosure relates generally to communication systems, and more specifically to an interactive multimedia collaboration platform with remote-controlled camera and annotation capabilities.

### Description of the Related Art

Computing systems can utilize communication networks to exchange data. In some implementations, a computing system can communicate with another computing system to share real-time or recorded communications, such as videos, images, voice, text, and so on.

### SUMMARY

In some aspects, the techniques described herein relate to a computer-implemented method for remote control of a live stream video field of view, the computer-implemented method including: under control of a handheld computing device including a video camera, an output, and one or more processors configured to execute specific computer-executable instructions, obtaining, using the video camera, video data representing a field of view of the video camera; sending a substantially live stream of the video data to a remote device; receiving, from the remote device while continuing to obtain video data representing the field of view, device movement data representing a desired movement of the handheld computing device to change the field of view; and presenting, using the output, a prompt to move the handheld computing device based on the desired movement.

In some aspects, the techniques described herein relate to a computer-implemented method, wherein receiving the device movement data representing the desired movement includes receiving data representing a magnitude and direction in which the handheld computing device is to be moved.

In some aspects, the techniques described herein relate to a computer-implemented method, wherein presenting the prompt includes displaying an arrow indicating a direction in which the handheld computing device is to be moved to satisfy the desired movement.

In some aspects, the techniques described herein relate to a computer-implemented method, further including: generating motion data using an inertial motion sensor; determining, based on the motion data, that movement of the handheld computing device has satisfied the desired movement; and ending presentation of the prompt.

In some aspects, the techniques described herein relate to a computer-implemented method, further including: analyzing the video data to determine a movement of the handheld computing device; determining that movement of the handheld computing device has satisfied the desired movement; and ending presentation of the prompt.

In some aspects, the techniques described herein relate to a computer-implemented method, further including presenting a second prompt in response to determining that movement of the handheld computing device has satisfied the desired movement.

In some aspects, the techniques described herein relate to a computer-implemented method, wherein presenting the second prompt includes generating haptic feedback.

In some aspects, the techniques described herein relate to a computer-implemented method, wherein presenting the second prompt includes displaying the second prompt.

In some aspects, the techniques described herein relate to a computer-implemented method, further including: receiving, from the remote device while continuing to obtain video data representing the field of view, capture setting data representing a capture setting of the video camera to be applied; and applying the capture setting to capture of the video data.

In some aspects, the techniques described herein relate to a computer-implemented method, wherein applying the capture setting includes changing one of: an exposure setting, a zoom setting, a color temperature setting, a flash setting, or a focus setting.

In some aspects, the techniques described herein relate to a computer-implemented method, further including: receiving, from the remote device while continuing to obtain video data representing the field of view, second capture setting data representing a second capture setting of the video camera to be applied, wherein the second capture setting is different from the capture setting; and applying the second capture setting to capture of the video data.

In some aspects, the techniques described herein relate to a computer-implemented method, further including: receiving, from the remote device while continuing to obtain video data representing the field of view, capture setting data representing a combination of capture settings of the video camera to be applied; and applying the combination of capture settings to capture of the video data.

In some aspects, the techniques described herein relate to a computer-implemented method, wherein applying the combination of capture settings includes changing two or more of: an exposure setting, a zoom setting, a color temperature setting, a flash setting, or a focus setting.

In some aspects, the techniques described herein relate to a computer-implemented method, further including: analyzing a frame of the video data to determine whether a region in the frame of video data includes potentially sensitive information; and in response to determining that the region includes potentially sensitive information, applying a visual mask to the region to generate anonymized video content.

In some aspects, the techniques described herein relate to a computer-implemented method, further including: storing at least a portion of the video data in a local data store of the handheld computing device; establishing a bidirectional audio communication connection with the remote device; receiving, from the remote device: playback data representing one or more playback commands for presentation of the video data; and annotation data representing one or more annotations to be presented with the video data; and presenting the portion of the video data from the local data store onto the output with the one or more annotations, wherein the portion of the video data is presented according to the one or more playback commands while maintaining the bidirectional audio communication connection with the remote device.

In some aspects, the techniques described herein relate to a computer-implemented method, further including: determining an aspect ratio of a display external to the handheld computing device based at least partly on one or more coordinates associated with the display in the video data; determining an orientation of the display in three-dimensional space at one or more time points in the video data based at least partly on the aspect ratio; and generating a substantially rectangular two-dimensional representation of the display at each of the one or more time points based on the orientation of the display at the one or more time points.

In some aspects, the techniques described herein relate to a computer-implemented method, further including: determining an aspect ratio of a display external to the handheld computing device based at least partly on one or more coordinates associated with the display in image data; determining an orientation of the display in three-dimensional space in the image data based at least partly on the aspect ratio; generating a substantially rectangular two-dimensional representation of the display based on the orientation of the display; and generate a cropped, perspective-transformed version of the image data based on the substantially rectangular two-dimensional representation of the display.

In some aspects, the techniques described herein relate to a computer-implemented method for remote control of a live stream video field of view, the computer-implemented method including: under control of a handheld computing device including a display and one or more processors configured to execute specific computer-executable instructions, receiving, from a remote device, video data representing a field of view of a video camera of the remote device; presenting the video data on the display; generating motion data representing a motion of the handheld computing device during presentation of the video data on the display; determining, based on the motion data, a movement of the handheld computing device; and sending, to the remote device, desired movement data representing a desired movement of the remote device.

In some aspects, the techniques described herein relate to a computer-implemented method, wherein generating the motion data includes generating the motion data using an inertial motion sensor of the handheld computing device.

In some aspects, the techniques described herein relate to a computer-implemented method, wherein generating the motion data includes generating the motion data based on an analysis of second video data obtained from a video camera of the handheld computing device.

### BRIEF DESCRIPTION OF THE DRAWINGS

Embodiments of various inventive features will now be described with reference to the following drawings. Throughout the drawings, reference numbers may be re-used to indicate correspondence between referenced elements. The drawings are provided to illustrate example embodiments described herein and are not intended to limit the scope of the disclosure. To easily identify the discussion of any particular element or act, the most significant digit(s) in a reference number typically refers to the figure number in which that element is first introduced.

FIG. 1 is a diagram of an illustrative computing environment in which aspects of interactive multimedia collaboration may be implemented according to some embodiments.

FIG. 2 is a block diagram of illustrative participant devices, including a capture device and a control device, and components thereof for providing aspects of interactive multimedia collaboration according to some embodiments.

FIG. 3 illustrates data flows and interactions with and between a capture device and a control device to provide remote control of the capture device according to some embodiments.

FIG. 4 is a top-down illustration of data flows and interactions with and between a capture device and a control device to provide remote control of the capture device according to some embodiments.

FIG. 5 illustrates data flows and interactions with and between a capture device and a control device during content consumption activities according to some embodiments.

FIG. 6 is a flow diagram of an illustrative routine for providing a live stream version of content optimized for transmission, and subsequently providing a full resolution version of the content or a portion thereof in response to a request from a participant device according to some embodiments.

FIG. 7 illustrates data flows and interactions with and between a capture device and a participant device during a live stream collaboration session where full resolution versions of content are requested and obtained according to some embodiments.

FIG. 8 is a flow diagram of illustrative operations that may be performed to generate non-destructive versions of annotated content in which the underlying content is maintained and accessible according to some embodiments.

FIG. 9 illustrates data flows and interactions with and between a control device and another participant device to generate, consume, and interact with non-destructive versions of annotated content according to some embodiments.

FIG. 10 is a flow diagram of an illustrative routine for automatically detecting and masking potentially sensitive information in video content according to some embodiments.

FIG. 11 illustrates example operations for capturing video content with potentially sensitive information and automatically detecting and masking the potentially sensitive information according to some embodiments.

FIG. 12 is a flow diagram of an illustrative routine for generating an improved view of a screen or printed content captured using a handheld device according to some embodiments.

FIG. 13 illustrates example effects of generating an improved view of a screen captured using a handheld device according to some embodiments.

FIG. 14 illustrates example processing of a display screen to generate a cropped, rotated, transformed-perspective view of a screen according to some embodiments.

FIG. 15 illustrates example user interfaces for discussions, chats, and cases according to some embodiments.

FIG. 16 is a block diagram showing relationships between—and organization of—discussions, chats, and cases according to some embodiments.

FIG. 17 is a block diagram of an illustrative computing device that may implemented aspects of the present disclosure according to some embodiments.

### DETAILED DESCRIPTION OF ILLUSTRATIVE EMBODIMENTS

The present disclosure relates to facilitating collaboration using visual content or multimedia content with a visual component, and to alternative presentation and documentation modes of exchanged discussions, visual content and multimedia content.

Conventional collaboration and communication systems and platforms may allow users to share video, images, and multimedia content. However, the content is typically unprocessed, or not processed in a way that improves certain aspects of information presentation and exchange. For example, conventional systems may compress video content to optimize transmission speed and facilitate substantially real-time communication. However, such compression can reduce the quality of content presented by the recipient devices, which can interfere with collaboration regarding fine details such as those in medical images. As another example, conventional systems may provide annotation features to markup content for viewing by recipients. However, the annotated content may suffer from the same relatively low quality as other substantially real-time content. In cases where high-quality content is annotated offline and shared asynchronously, the underlying non-annotated content may not be easily obtained if desired by recipients, or may even be irreversibly masked by the overlying annotation and therefore lost. As a further example, some video capture systems provide optical image stabilization to counteract the effects of camera movement, or use ultra-wide angle camera lenses and selective cropping to give the effect of a camera that pans to keep a moving human subject in a frame. However, such techniques do not address the issues presented by a moving camera that captures video of a stationary information-rich object (e.g., a screen, computer monitor or printed picture or medical image) for which camera motion and changes in viewing perspective can hinder the clear understanding and sharing of information with collaborators.

The issues and challenges noted above, among others, can interfere with effective collaboration, and particularly collaboration involving fine visual details such as medical images, procedures, documents, and the like. Advantageously, aspects of the present disclosure address some or all of these issues, among others, using an interactive multimedia collaboration platform with remote-controlled camera and annotation capabilities that preserve, enhance, and organize the presentation of fine visual details and other information communicated among participating collaborator devices.

Some aspects of the present disclosure relate to an improved method of capturing and sharing visual content or multimedia content (e.g., a live audiovisual feed) with collaborators. The content may be provided substantially as-captured, or it may be processed first to remove sensitive information or improve recipients' experience.

In some embodiments, a capture device may store a high-quality version of content that is being transmitted in a lower-quality form to recipients (e.g., content that is compressed to optimize transmission speed). This can provide a substantially live stream to recipients, and also allow participants to request high-quality versions of frames or video segments that are of particular interest, such as those with fine details that are difficult to visualize or that may be lost altogether in the lower-quality live stream form. For example, a recipient may be viewing a stream, generated by a capture device, of a medical image or medical procedure. The recipient may wish to take a closer look at a particular portion of the stream, such as an individual frame or portion of video. The recipient may request and receive, from the capture device, a high-quality version of the content of interest, and the capture device may provide the high-quality version from its own local storage. When the recipient receives the high-quality version of requested content, the recipient may be enabled to interact with the content as desired, such as by pausing, rewinding, fast forwarding, zooming, annotating, etc. Advantageously, the view of high-quality content on the recipient device may be replicated on the capture device while maintaining a live audio communication channel between the capture device and recipient device. For example, the capture device may present high-quality content from its own local storage, and may replicate the view from the recipient device based on interaction data received from the recipient device such as time stamps, zoom details, playback commands, annotations, and the like. Thus, the two devices may present high-quality content from their respective local storages, mitigating any issues with live stream video communications between the devices, while also permitting live stream audio communication (which may include a small or “thumbnail” lower resolution live video stream displayed on a portion of the screen), and replication of what is presented on the visual displays of the devices.

In some embodiments, both the capture device and the recipient device may send interaction data (time stamps, playback commands, annotations, and the like) to each other, enabling bi-directional interactive review of content such as a video segment, which was shared among the two devices and is stored in the local storage of both devices. Enabling bi-directional interactive review of content, a command sent by any device to the other device may be simultaneously performed by both devices. In some cases, where multiple commands are sent, the commands may be prioritized according to a predetermined order or hierarchy (e.g., having the last command overrule all previous commands, having a command associated with a higher level of a hierarchy overrule a command associated with a lower level, having a command from a device associated with a higher level of a hierarchy overrule a command from a device associated with a lower level). As an example, any device may send a “play” command thereby effecting playing of the video content on both devices, and thereafter any device may send a “pause” command, effecting pause of the video content at the same frame in both devices. Alternatively, one of the two devices may be associated with a higher level of a control hierarchy than the other device, and may pause the video such that the other device is not permitted to resume playback due to the other device being associated with a lower level of the hierarchy, or the device associated with the higher level of the hierarchy may have the sole ability to erase an annotation, and the like. It is appreciated that the above examples refer to two communicating devices, but may be generalized and applicable to more than two devices, thereby allowing group communication. As an example, the capture device may send the high-quality content as explained above to more than one recipient device simultaneously. As another example, a group of more than two devices may review simultaneously a video segment which was previously sent to all group devices by any of the group devices and is stored in the local storage of each of the devices in the group, as explained above. A predetermined command order or hierarchy may be defined for the group-review, for example the last command sent by any group device will be effected in all group devices, overriding previous commands as applicable. Alternatively, a particular device in the group, for example the device inviting or initiating the group review or discussion, may be associated with a higher level of the control hierarchy than other devices in the group, and as such may have the power to erase annotations made by other devices, pause the video at a certain frame such that other devices cannot resume playback, etc.

In some embodiments, a capture device may process captured video content to smooth camera motion and flatten the perspective of generally rectangular information-rich objects, such as documents, printed images, or display screens. Such smoothing and perspective transformation can be particularly advantageous when the capture device is a handheld device, such as a smart phone or tablet or a portable digital camera, and the information-rich object is a substantially stationary object. Moreover, a smoothed, perspective-transformed view of such an object can be cropped to provide a full-frame or substantially full-screen view of the object to further improve the view provided to recipients. Advantageously, capture parameters such as brightness, focus, contrast and the like may be determined, balanced, averaged or optimized based on the visual content within the cropped object, disregarding the visual content outside the cropped object, thereby providing an optimized cropped visual content.

In some embodiments, a capture device may automatically detect and mask sensitive data (e.g., personally-identifiable data, health data, human face, etc.) on screens prior to providing video of the screens to recipients. For example, the capture device may use a machine learning model to identify, within a video, regions of potentially sensitive information such as a personally-identifiable information, personal health information, or the like. When such regions are detected, the capture device may mask the regions so that the sensitive information is not exposed to recipients. In addition, capture device users may manually indicate regions of potentially sensitive information to be masked, or masked regions that do not include potentially sensitive information and therefore should not be masked. Feedback regarding such user interactions can be used to retrain or otherwise update the machine learning model to continuously improve sensitive information detection performance, in terms of both accuracy and recall.

Additional aspects of the present disclosure relate to providing recipients of video content with the capability to remotely control aspects of content capture. In some embodiments, a recipient may physically change the orientation of—or otherwise manipulate—the device on which they are viewing a live video stream. An instruction to perform a corresponding reorientation or manipulation may be presented on the capturing device. For example, a recipient may wish to pan in a particular direction to adjust the field of view captured in the video stream. When the recipient performs such a movement with the recipient device, the recipient device may generate and send an instruction to the capture device regarding the desired movement to be made with the capture device. The capture device may then present, to a user of the capture device, a prompt regarding the desired movement, such as by displaying an arrow in the direction in which the capture device is to be moved or reoriented. In addition to the particular type of desired movement, data regarding the magnitude of the movement may also be sent to the capture device so that when the capture device has been adjusted to provide the desired field of view, an indication may be presented to the user of the capture device. For example, haptic feedback may be provided, a visual prompt to move the capture device may be removed from the display screen, or a visual confirmation may be displayed. In this way, the user of the capture device can be prompted to perform a particular type and magnitude of action, and be informed when the desired action has been completed (e.g., so the user doesn't overmanipulate the device).

Further aspects of the present disclosure relate to generating and managing annotations to content that may be shared with recipients in a way that permits access to the original or otherwise non-annotated content. Such annotations may be referred to as “non-destructive” annotations when their creation does not alter the underlying content, and the unaltered underlying content may be accessed, viewed, annotated differently, or the like. As used herein, the terms “annotation” and “annotated content” refer to content that is collected, composed, altered, marked up, or otherwise modified or prepared for presentation. Some annotations are composed of a single static image (e.g., a frame of video) to which various markups or modifications have been applied. Such annotations may be referred to as “snapshots.” Some annotations are dynamic in the sense that presentation (e.g., audio and/or video output) changes over a period of time from start to end. Such annotations may be referred to as “narrations” or “video-based annotations.” For example, a video-based annotation may be composed of one or more images, portions of video, or a combination thereof presented in a predetermined user-defined sequence. Video-based annotations may also include markups, viewing manipulations, audio tracks (e.g., user speech), and the like.

In some embodiments, a capture device may generate content by capturing video or an image of a live human subject, a screen, a printed image, a document, another subject, or some combination thereof. The content may be optionally augmented using one or more methods to improve viewing and consumption, such as by smoothing camera motion and flattening the perspective of generally rectangular information-rich objects, detecting and masking potentially sensitive information, and the like. The content—whether raw content captured by the capture device, augmented content that has been augmented using one or more methods, or the like—may then be modified for presentation. For example, content may be modified by drawing on the content (e.g., using a finger or stylus), adding textual markups (e.g., by typing or using speech recognition), adding audio to the content (e.g., by recording a user's spoken words), altering playback or presentation of the content (e.g., pausing, rewinding, zooming, cropping, etc.), combining presentation of multiple content items according to a timeline, or by performing other manipulations of or additions to content. To maintain separation between the annotations and the underlying content such that the annotations are non-destructive, the annotations may be defined or referenced in annotation metadata that may be provided with the underlying content while remaining physically separate (e.g., in a separate physical file, in a header of the content file separated from the content itself, etc.). Annotation metadata may represent coordinates, colors, shapes, and other properties of on-screen annotations and the timestamps at which they are to be displayed, coordinates and content of typed annotations and the timestamps at which they are to be displayed, audio and the timestamps at which it is the be presented, other metadata, or any combination thereof as desired. Annotations may be generated on the capture device itself, or on another device after being shared with the other device. Advantageously, the non-destructive nature of the annotations can allow recipients to view and interact with the annotated content as it was generated by the annotating user, while also allowing recipients to access the underlying non-annotated content to view it unannotated, add new annotations, alter the annotations generated by the first annotating user, or any combination thereof.

Still further aspects of the present disclosure relate to organizing communications among collaboration participants such that they are searchable and accessible under top-level tiers of multiple separate message hierarchies. In some embodiments, communications among collaboration participants may include any number of content objects such as messages (e.g., text-based messages), images, videos, annotated content, and the like. One hierarchy may organize such communications according to a group of participants, such that each unique group of participants is a top level of the participant-based hierarchy and the content objects shared among the group of participants is beneath a single top level of the participant-based hierarchy. A group of communications in this hierarchy may be referred to as a “chat,” and may be defined in terms of the set of users participating. Another hierarchy may organize communications and other content according to subject matter. For example, a particular medical case may serve as a top-level of a case-based hierarchy. Content objects associated with the medical case may be beneath the top level of the case-based hierarchy, without regard to which users are participating in individual discussions. In this way, users may access content in a variety of ways, depending upon whether they are searching according to the participants of a conversation or subject matter of the conversation.

Advantageously, assignment of particular chats to particular cases may be made in a manner that maintains separation among each chat and maintains separation among each case, while facilitating discussion of cases in chat conversations and facilitating access to chat conversations alongside case files and other attachments. Thus, a complete conversation history among a particular group of users may be preserved separately from a complete history of a particular case, while allowing users to switch back and forth between chat-centric and case-centric views of information related to both a chat and a case. For example, it can be important to maintain the history, chronology and completeness of data (messages, attachments) related to a medical case so that users can refer back to any symptoms, medications prescribed, recommendations made, referrals given, and follow-ups conducted.

Various aspects of the disclosure will be described with regard to certain examples and embodiments, which are intended to illustrate but not limit the disclosure. Although aspects of some embodiments described in the disclosure will focus, for the purpose of illustration, on particular examples of content, communications, annotations, narration, and processing operations, the examples are illustrative only and are not intended to be limiting. In some embodiments, the techniques described herein may be applied to additional or alternative types of content, communications, annotations, narration, and processing operations. Additionally, any feature used in any embodiment described herein may be used in any combination with any other feature or in any other embodiment, without limitation.

### Example Network-Based Collaboration Environment

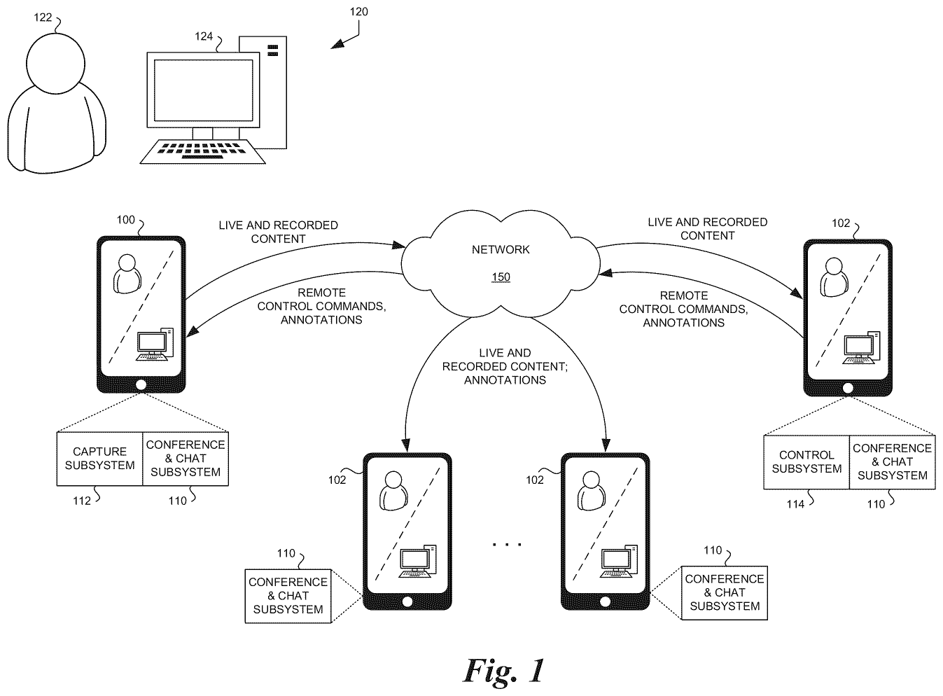

FIG. 1 illustrates an illustrative collaboration environment including multiple user devices in communication via one or more communication networks. In the example shown, a capture device 100 is capturing visual content, such as video or images, and providing the visual content to participant devices 102 via communication network 150.

Communication network 150 (also referred to simply as “network 150” for brevity) may be a publicly-accessible network of linked networks, possibly operated by various distinct parties, such as the internet. In some embodiments, network 150 may be or include a private network, personal area network, local area network, wide area network, cable network, satellite network, cellular telephone network, or a combination thereof, some or all of which may or may not have access to and/or from the internet.

The individual user devices, including capture device 100 and participant devices 102, may be any of a wide variety of computing devices, including personal computing devices, laptop computing devices, tablet computing devices, electronic reader devices, wearable computing devices, mobile devices (e.g., smart phones, media players, handheld gaming devices, etc.), and various other electronic devices and appliances. In some embodiments, any or all of the user devices may be a handheld computing device.

With reference to an illustrative embodiment, a collaboration conversation participant may operate a user device, such as capture device 100, to capture visual content of one or more subjects 120 and provide the visual content to one or more participant devices 102 via the network 150. In the illustrated embodiment, the capture device 100 is a handheld device, such as a smart phone or tablet computing device. The user of the capture device 100 may launch specialized application software, such as a conference and chat subsystem 110 configured to provide various collaboration functionalities described herein, such as facilitating live virtual conferences and asynchronous chat conversations. The capture device 100 may also include a capture subsystem 112 that is configured to control operation of a camera of the capture device 100 (e.g., a video camera, still image camera, or camera configured to capture both video and still images), apply any desired processing, and provide the captured content to participant devices 102. As shown illustratively, the capture device 100 may capture visual content regarding various subjects in a field of view of a camera of the capture device 100. For example, the capture device 100 may capture visual content of a living subject, such as a human 122. As another example, the capture device 100 may capture visual content of a non-living subject, such as a display screen 124 of a computing system or other visual presentation system.

The participant devices 102 may each also include specialized application software, such as the conference and chat subsystem 110 configured to provide various collaboration functionalities described herein. The conference and chat subsystem 110 may allow the participant devices 102 to present and interact with content from a capture device 100, communicate with other participant devices 102, and the like. Some or all of the participant devices may also include a control subsystem 114 that facilities remote control, by a participant device, of the capture device 100 as described in greater detail below.

Although FIG. 1 shows only a single capture device 100 having a capture subsystem 112 and a single participant device having a control subsystem 114, the example is provided for purposes of illustration only and is not intended to be limiting or required. In some embodiments there may be more than one capture device 100, more than one participant device 102 with a control subsystem 114, only a single participant device, 102, etc. In some embodiments, each device of the collaboration environment may include a conference and chat subsystem 110, a capture subsystem 112, and a control subsystem 114. In some embodiments, one or both of the capture subsystem 112 or control subsystem 114 may be integrated with the conference and chat subsystem 110 such that a single executable software application provides the functionality of each subsystem or subsets thereof.

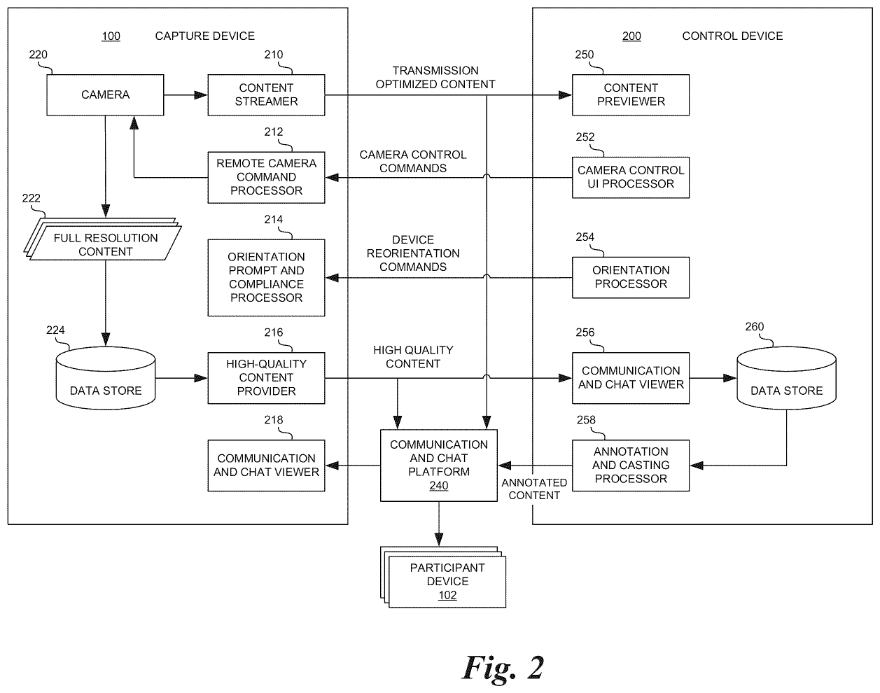

FIG. 2 illustrates components of—and interactions between—various devices that may implement various features described herein, including a capture device 100 and a control device 200. The control device 200 may be a particular participant device 102 that is configured to control—or is in the process of controlling—aspects of content capture and other functionality of the capture device 100. In the description that follows, it will be assumed that any participant device 102 may be a control device 200 depending upon the particular collaboration session that is occurring, interactions that are occurring, and so on. However, the description is provided for purposes of illustration only, and is not intended to be limiting or required. In some embodiments, the control device 200 may be considered to be different from a participant device 102, regardless of the interactions that are occurring during a collaboration session. For example, specialized application software may be installed on a control device 200 to facilitate controlling aspects of content capture and other functionality of the capture device 100, and the participant devices 102 may not have such application software installed.

As shown in FIG. 2, a capture device 100 may have a camera 220 to capture visual content such as video or images, a data store 224 to store content generated by the camera 220, a content streamer 210 to manage sending live stream content to other devices participating in a live collaboration session, a remote camera command processor 212 to process and apply camera control commands received from other devices participating in a live collaboration session, an orientation prompt and compliance monitor 214 to process device reorientation commands and determine reorientation compliance, a high-quality content provider 216 to provide high quality content (e.g., full resolution content 222 as generated by the camera 220) in response to requests from other devices participating in a collaboration session (e.g., control device 200, a participant device 102), and a communication and chat viewer 218 to facilitate other communications and interactions, including communications and interactions outside of a live collaboration session. Examples of the functionality provided by the components are described in greater detail below. The individual components may be implemented in hardware, or as a combination of hardware executing application software. For example, the capture subsystem 112 shown in FIG. 1 may include executable software that programs hardware processors and other hardware components of the capture device 100 to operate the camera 220 and provide the functionality of the remote camera command processors 212 and the orientation prompt and compliance monitor 214. As another example, the conference and chat subsystem 110 may include executable software that programs hardware processors and other hardware components of the capture device 100 to provide the functionality of the content streamer 210, the high-quality content provider 216, and the communication and chat viewer 218.

As shown in FIG. 2, a control device 200 may include a content previewer 250 to provide a view of live stream content received from the capture device 100, a camera control user interface (UI) processor 252 to respond to various UI control interactions and send camera control commands to a capture device 100, an orientation processor 254 to respond to various movements of the control device 200 and send device reorientation commands to the capture device 100, a communication and chat viewer 256 to facilitate other communications and interactions, including receipt of high quality content that is then stored in a local data store 260, and an annotation and casting processor 258 to facilitate generation of annotations and communication of the annotations to other devices. The functionality provided by the components is described in greater detail below. The individual components may be implemented in hardware, or as a combination of hardware executing application software. For example, the control subsystem 114 shown in FIG. 1 may include executable software that programs hardware processors and other hardware components of the control device 200 to provide the functionality of the content previewer 250, the camera control UI processor 252, and the orientation processor 254. As another example, the conference and chat subsystem 110 may include executable software that programs hardware processors and other hardware components of the control device 200 to provide the functionality of the communication and chat viewer 218, and annotation and casting processor 258.

In some embodiments, a communication and chat platform 240 may be implemented as a central server separate from the capture device 100, control device 200, and other participant devices 102. The communication and chat platform 240 may provide storage and organization for asynchronous collaboration, as described in greater detail below.

### Interactive Remote Control of Capture Device

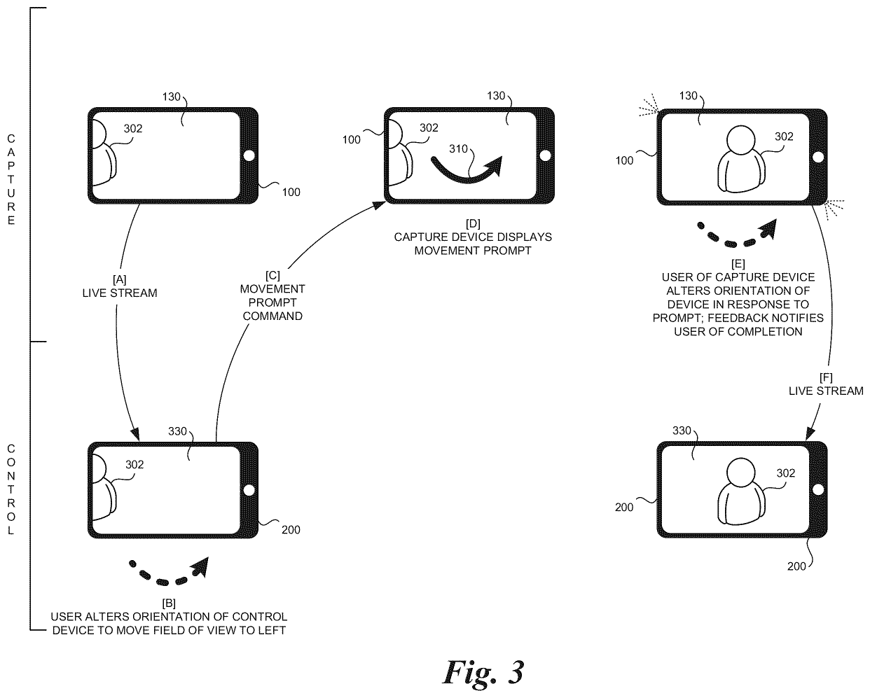

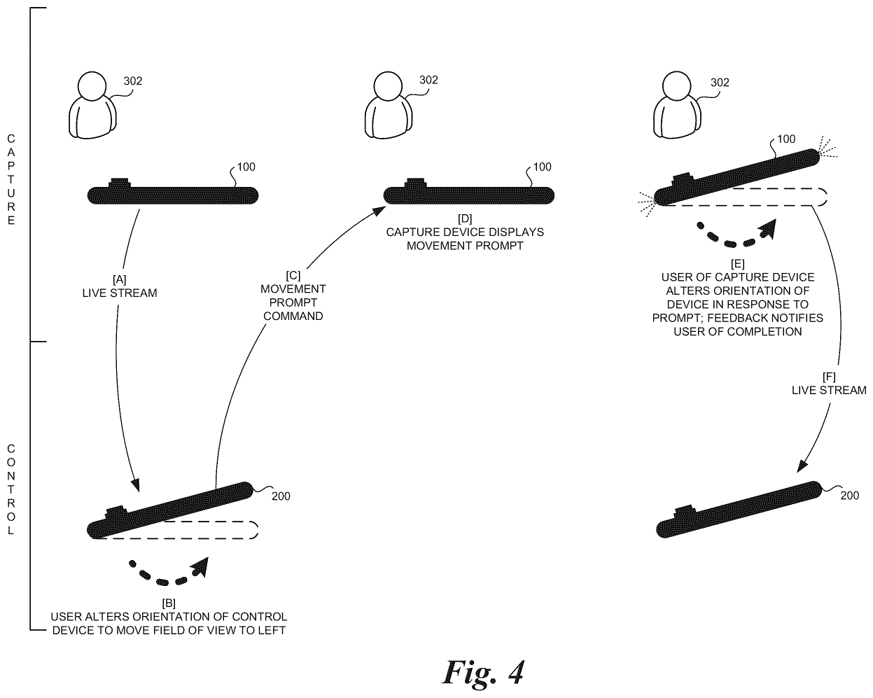

FIGS. 3 and 4 illustrate example interactions and data flows between a capture device 100 and a participant device 102 in connection with remote control of content-capture operations performed by the capture device 100. In this context, because the participant device 102 is remotely controlling aspects of the capture device 100, the participant device will be referred to as a control device 200.

At [A], the capture device 100 may transmit visual content to the control device 200. The visual content may be transmitted as a live stream of content that is available for presentation by the control device 200 in real time, as the content is generated by the capture device 100. In some embodiments, the visual content may also be transmitted to one or more additional participant devices 102. However, for simplicity the discussion that follows focuses on the transmission to only control device 200.

As used herein, the term “real time” is used according to its usual and customary meaning in computing and networking, and refers to the effectively contemporaneous nature of the events being described. In computing environments, events in different locations on a network, or different events within a single computing device, rarely occur at exactly the same time. There may be an offset in timing due to latencies inherent in network communications, computer processing, and the like. Thus, the term “real time” does not necessarily equate to “exactly the same time,” but rather the observed effect of two or more events occurring at approximately the same time for practical purposes and when factoring in network communication and processing. Thus, the concept of “real time” is often referred to herein as “substantially real time.”

As used herein, the terms “streaming content,” “content stream” and “stream” are used according to their usual and customary meaning in computing and networking. The terms refer to content that is delivered in substantially real time as it is created and/or content that is presented in substantially real time as it is received, without first requiring transmission of a complete file for the entirety of the content item.

As used herein, the term “live stream” is used according to its usual and customary meaning in computing and networking, and refers to streaming content that is presented in real time or substantially real-time as it is being recorded or otherwise generated. The concept of “live stream” content may be understood by way of contrast with “on demand” content, the presentation of which may also be accomplished through a stream. Whereas both “live stream” and “on-demand” streaming content may be delivered and simultaneously (or substantially simultaneously) presented by a participant device without requiring a complete download of the content file being presented, “on-demand” streaming content is streamed from a content data store after it is created and stored. For example, on-demand streaming content may be available for streaming a significant period of time after its creation (e.g., days or years later), where the period of time is not solely due to networking and processing latencies. In contrast, live stream content is delivered and presented in substantially real time as it is being created, though in some cases delivery of live stream content may experience delays in delivery to devices for presentation (e.g., delays of seconds or minutes due to networking or computing latencies, substantial processing, and/or implementation of moderation polices).

As shown in FIGS. 3 and 4, the live stream presented on the display 330 of the control device 200 reflects the content captured and presented on the display 130 of the capture device 100. In this example, the live stream is a representation of a field of view of a camera of the capture device 100, which includes a human subject 302 partially outside the field of view.

At [B], a user of the control device 200 may determine that it is desirable to reorient the capture device 100 to adjust what is visible in the field of view of the camera of the capture device 100 and therefore in the live video presented on the control device 200. Instead of—or in addition to—verbalizing commands to reorient the capture device 100, the user of the control device 200 may cause the control device 200 to enter a camera guidance mode in which motion of the control device 200 is sensed, and device movement data is generated. For example, the user of the control device 200 may activate a user interface option to enter the camera guidance mode, and deactivate the user interface option when the user is finished remotely controlling the movement of the capture device 100.

Once the camera guidance mode has been activated, the user of the control device 200 may reorient the control device 200 in a way that would adjust the field of view if what was presented on the display 330 was being generated by a camera of the control device 200. The control device 200 may have one or more motion sensors that sense motion of the control device 200 and generate data representing the motion. For example, the control device 200 may include an inertial measurement unit (IMU) with one or more accelerometers, gyroscopes, other motion sensors, or some combination thereof. The IMU of the control device 200 may generate motion data representing the magnitude and direction of change that has been sensed.

In some embodiments, one or more other methods to determine the motion of the control device 200 may be used instead of, or in addition to, use of a physical motion sensor such as an IMU. For example, an object or set of objects in the environment of the capture device 100 may be detected, and the size, distance, or other factors for determining the relative position of the object(s) with respect to the control device 200 in space may be estimated. The detection and estimation may be performed using data from one or more line of sight sensors, such as a LiDAR sensor, a user-facing camera, a rear-facing camera, another sensor, or some combination thereof. When the user of the control device 200 reorients the control device 200 in space, the relative position of the detected object(s) will change. The difference in relative position of the object(s) before and after the user reorients the control device 200 can be calculated, and the magnitude and direction of change in orientation of the control device 200 may be determined based thereon. Motion detected in the way can be used to confirm motion detected using a physical motion sensor (e.g., an IMU), or may be used instead of a physical motion sensor.

In the illustrated example, the human subject 302 is partially out of the left side of the field of view, and the user of the control device 200 may desire to reorient the capture device 100 such that the human subject 302 is fully into the field of view. Thus, the user may tilt or rotate the control device 200 such that the right-side edge moves away from the user and/or the left-side edge moves closer to the user, as indicated by the dashed arrow. FIG. 4, which is a top-down view of the interactions illustrated in FIG. 3, shows the effect of the movement of the control device 200 on the orientation of the device. The physical motion of the control device 200 may be sensed by the IMU (or by other motion sensing method), which generates motion data that represents the physical motion.

At [C], the control device 200 may transmit device movement data to the capture device 100 regarding the movement of the control device 200 sensed at [B]. Put differently, the control device 200 may transmit device movement data to the capture device 100 regarding movement to be taken by the capture device 100. The device movement data may be the motion data generated by the IMU of the control device 200, or movement data based thereon. The data may be sent over a same bidirectional connection that is being used to send and receive video data, audio data, and other data regarding the active conference. In some embodiments, a separate connection may be established, or separate multiplexed channel over the same connection may be used, to transmit the device movement data to the capture device 100.

At [D], the capture device 100 may process the device movement data and present a prompt to the user of the capture device 100. The capture device 100 may present the prompt visually, audibly, via some other modality, or in a combination of modalities. In some embodiments, if the device movement data represents motion in three-dimensional space to be applied to the capture device 100, the capture device 100 may convert the device movement data into a visual representation for display on the display 130 of the capture device 100. In the illustrated example, the capture device 100 presents a prompt 310 in the form of an arrow indicating the direction in which the capture device 100 is to be moved.

At [E], the user of the capture device 100 may reorient or otherwise move the capture device 100 in response to presentation of the prompt 310. As the user moves the capture device 100, the field of view changes accordingly and the change is reflected in the content stream that is provided to the control device 200 at [F]. FIG. 4, which is a top-down view of the interactions illustrated in FIG. 3, shows the effect of the movement of the capture device 100 on the orientation of the device.

The capture device 100 may have one or more motion sensors that sense motion of the capture device 100 and generate data representing the motion. For example, like the control device 200, the capture device 100 may include an IMU with one or more accelerometers, gyroscopes, other motion sensors, or some combination thereof. The IMU of the capture device 100 may generate motion data representing the magnitude and direction of movement that has been sensed. The capture device 100 may evaluate the motion data representing the magnitude and direction of the movement against the device motion data received from the control device 200. When the capture device 100 has completed movement that is equal to (or, in some embodiments, within a threshold measurement of) the movement represented by the device motion data, the capture device 100 can indicate completion of the requested movement. In some embodiments, indicating completion of the movement may be presented visually (e.g., by removing a prompt 310), audibly (e.g., presentation of a bell), haptically (e.g., activation of a motor to vibrate the capture device 100), or by some combination thereof. Completion of the desired movement is expected to result in adjusting the field of view of the camera, as represented in the video stream, in a way that is sufficient to achieve the desired result of the control device 200. In the illustrated example, the human subject 302 has been moved fully into the center of the field of view presented on the display 330 of the control device 200.

In some embodiments, from time to time or when the user of the control device 200 exits the camera guidance mode, the control device 200 may normalize the accumulated changes in device movement to a standard IMU measurement. The control device 200 may generate and send to the capture device 100 device movement data that is not to be acted upon in the same manner as described above. The capture device 100 may receive the device movement data and use it to calibrate against represented motions sensed by the local IMU of the capture device 100.

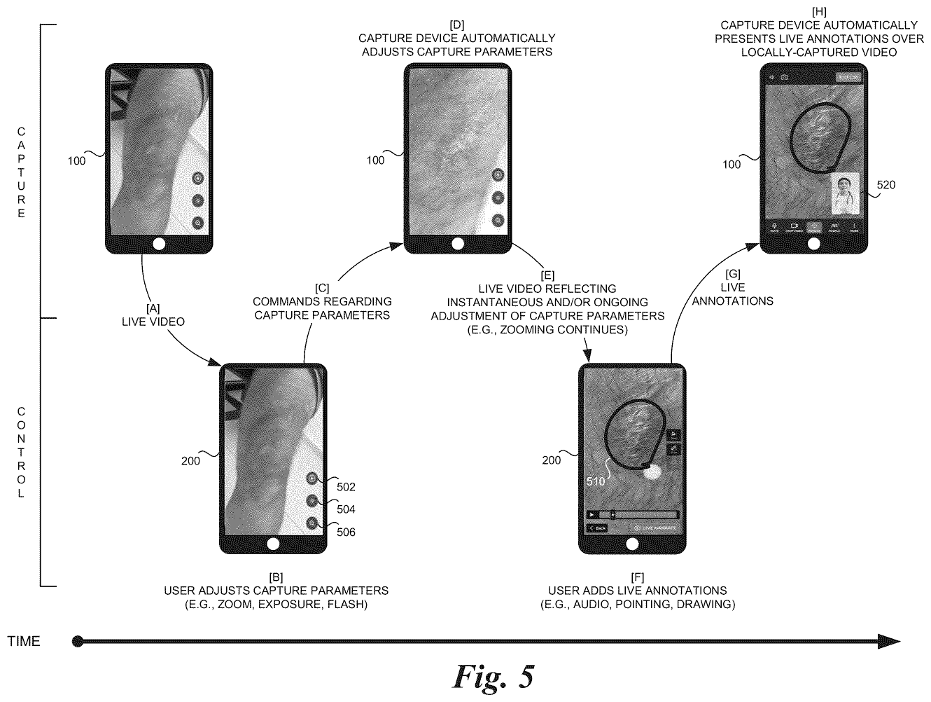

FIG. 5 illustrates additional or alternative commands that may be initiated remotely by a control device 200 to alter the capture or generation of video content by the capture device 100 over a period of time, as indicated by the timeline. As shown, at [A] the capture device 100 may provide substantially live stream video content to the control device 200, which presents the video content on a display of the control device 200. In some embodiments, the control device 200 may also present a set of user interface controls including, but not limited to, exposure setting, zoom, color temperature, flash on/off, focus point, other capture parameters, or any combination thereof.

In the example shown in FIG. 5, the display of the control device 200 is presenting user interface control 502 for controlling the flash of the capture device 100, user interface control 504 for controlling the exposure of the capture device 100, and user interface control 506 for controlling the zoom of the capture device 100. In some embodiments, one or more user interface controls may be presented for predetermined combinations of capture parameters depending upon the subject of the video content. For example, different combinations of capture parameters may be provided for optimized capture and viewing of live humans, wounds, screens, printed images, or other types of content. Thus, a user may activate a single user interface control and cause adjustment or implementation of multiple corresponding capture parameters.

At [B], a user may activate one or more of the user interface controls to adjust capture parameters of the capture device 100. For example, the user may tap on a corresponding icon. At [C], the control device 200 can send one or more commands regarding setting or modifying capture parameters to the capture device 100. For example, a command may have a name or include an identifier of the particular capture parameter to be set, the value or selection to which the capture parameter is to be set, other information, or a combination thereof.

At [D], the capture subsystem 112 may respond to the command(s) by adjusting one or more capture parameters (e.g., zoom in, change exposure), depending upon the commands received from the control device 200. In some embodiments, some capture parameter adjustments may be applied and reflected in live video instantaneously, while others may occur over a period of time. For example, application of exposure settings may be instantons or substantially instantaneous, while application of a change in zoom may occur over a period of time as an optical or digital zoom feature of the capture device 100 is operated. In the illustrated example, the user of the control device 200 may have activated the zoom user interface control 506 and selected a “zoom in” command to enlarge the captured content and, in response, the capture device 100 may automatically adjust the zoom parameter of its camera to increase the degree of zoom to the level commanded by the control device 200. The increased zoom may then be reflected in the live video that continues to be sent to the control device 200 at [E]. Because the interface of the control device at [F] is shown at a time that is after [D] and [E], a larger degree of zoom is shown to reflect the continuing zoom that has occurred on the capture device 100 to reach the desired degree of zoom.

In some embodiments, instead of or in addition to providing remote control of camera functions, a user of the control device 200 may markup or otherwise modify the presentation of content to create a live annotation. The markup or other modifications may be applied to the video content on a non-destructive basis. For example, markups may be saved as metadata that may be dynamically applied to the presentation of the video content without permanently altering the video content itself.

In the example shown in FIG. 5, a user of the control device 200 may add a markup 510 to the video content at [F]. The markup 510 may be drawing (e.g., drawn with a stylus or a finger), typed textual content, or another visual augmentation added to the display of the video content. The live annotation(s) may be provided to the capture device 100 (and, in some cases, one or more other participant devices) at [G]. For example, to preserve the underlying content and not alter it on a permanent basis, annotation metadata regarding the annotations (e.g., identifications of frames or video portions to be presented, degrees of zoom to be applied, coordinates of viewports to be displayed, vector graphics instructions for drawn or otherwise added annotations, timestamps at which annotations or other display aspects are to be presented, etc.) may be generated and provided by the control device 200 to the capture device 100 (and, in some cases, other participant devices). The annotation metadata may be provided separately from other collaboration session content, such as live bidirectional audio, live video from the control device 200 such as video from a user-facing camera of the control device that may be presented by the capture device 100 in an inner window 520 along with the annotated content (and, in some cases, other participant devices), and the like. For example, the annotation metadata may be provided over a separate physical connection, over a separate logical multiplexed connection with other content over a single physical connection, interleaved with other content, or using some other technique for transmission of multiple logically separate streams or items of data. Advantageously, such separation of annotation metadata can facilitate continued spoken communication between the users of the capture device 100 and control device 200 (and, in some cases, other participant devices) during presentation of—and interaction with—annotated content.

The capture device 100 may use the annotation metadata at [H] to present the annotations in an identical or substantially similar manner as they are created on the control device 200. For example, the capture device 100 may open the annotation metadata, determine which content to be loaded and presented from local storage (e.g., a frame or video segment), apply a specified degree of zoom, display a specified viewport, overlay vector graphics for drawn or otherwise added annotations, and the like at the timestamps included in the annotation metadata. Because the annotation metadata is provided as metadata separate from underlying video content, and because the underlying video content may be stored in unaltered form locally on the capture device 100 (and the control device 200), the user of the capture device 100 may interact with the content in various ways. For example, the user of the capture device 100 may access the underlying content. As another example, the user of the capture device 100 may add annotations. As a further example, the user of the capture device 100 may alter or remove the annotations made on the control device 200. As an additional example, the user of the capture device 100 may initiate sending altered or additional annotations to the control device 200 (and, in some cases, other participant devices). As yet a further example, any annotation command generated locally by any of the capture device 100 or control device 200 (such as adding or removing a markup, changing a display parameter, or the like) is displayed on the generating device and is transmitted simultaneously to the other device and displayed on it, such that the two devices (or any number of devices participating in a group communication) display the same content, synchronously. In such an embodiment, a predetermined hierarchy of executing commands by the participant devices may be dictated, for example the last command overrules all previous commands. For example, the predetermined rule may be that an annotation generated by a participant device is displayed on all devices in addition to all already existing annotations, or instead of all existing annotations which are removed once a new annotation is generated and displayed.

The creation and sharing of non-destructive annotations are described in greater detail below.

### Live Collaboration

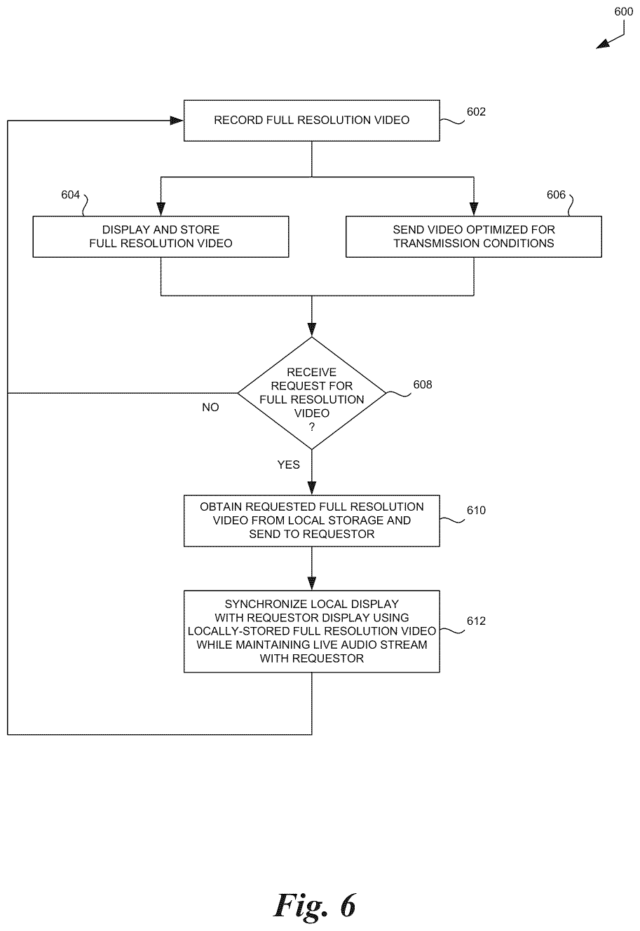

FIG. 6 is a flow diagram of an illustrative routine 600 for managing aspects of a live collaboration session based on video content captured by a capture device 100. A live collaboration session may also be referred to as a live conference. The capture device 100 may capture or otherwise generate full resolution content (e.g., content at a highest resolution provided by a camera of the capture device 100), and a user of the capture device 100 may desire to share the content with one or more participant devices. Advantageously, the capture device 100 may initially send a transmission-optimized version of captured/generated content (e.g., a down-sampled or compressed version) to the participant device 102, and later send a full resolution version of the captured/generated content on demand, in response to a request from the participant device 102.

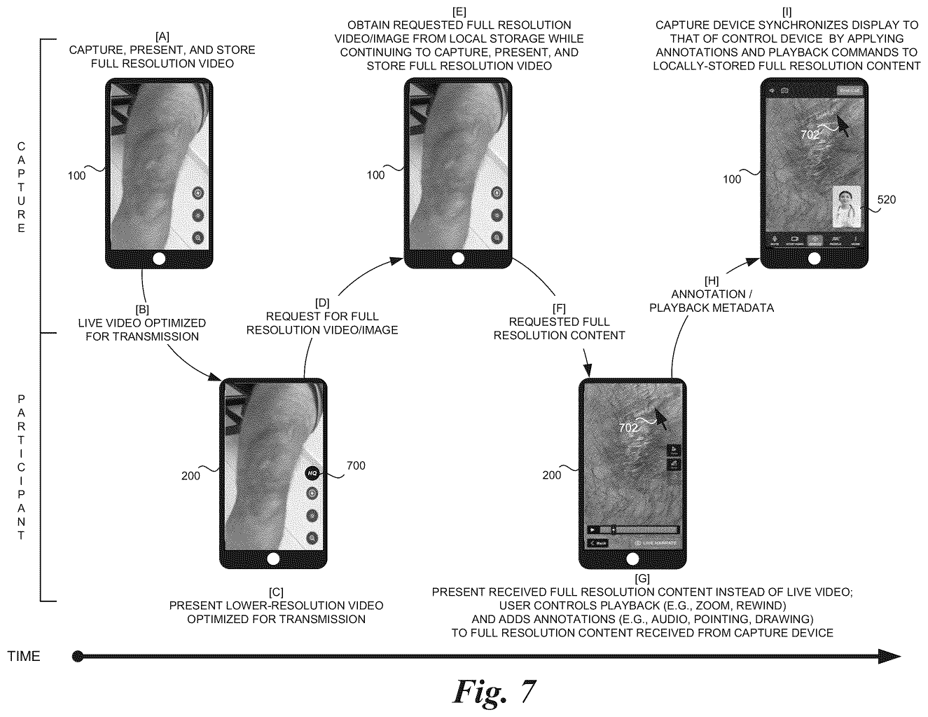

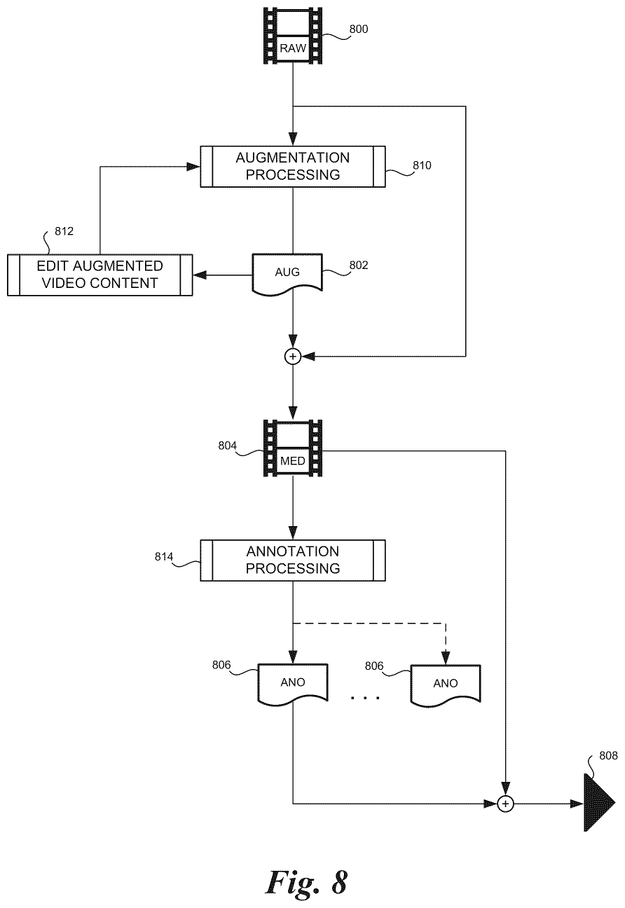

Portions of FIG. 6 will be described with further reference to the example video content and interactions therewith shown in FIGS. 7 and 8. Although the examples shown in FIGS. 7 and 8 and described below relate to video content of a live subject (e.g., a medical patient), the example is provided for purposes of illustration only and is not intended to be limiting or required. In some embodiments or in some situations, video content may include video of a screen, a printed image or document, or some other subject of a live collaboration session.

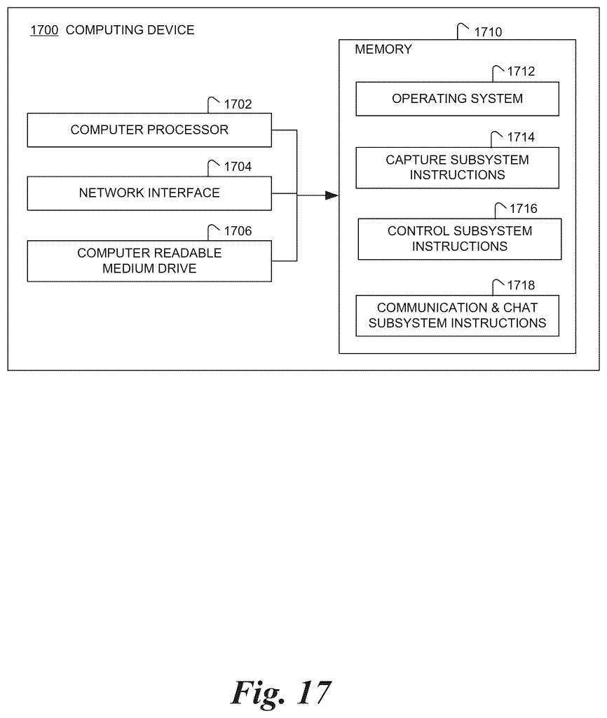

The routine 600 may be computer-implemented method that begins in response to an event, such as when the capture device 100 begins capturing video content. When the routine 600 is initiated, a set of executable program instructions stored on one or more non-transitory computer-readable media (e.g., hard drive, flash memory, removable media, etc.) may be loaded into memory (e.g., random access memory or “RAM”) of a computing device, such as the computing device 1700 shown in FIG. 17 and described in greater detail below. In some embodiments, the routine 600 or portions thereof may be implemented on multiple processors, serially or in parallel.

At block 602, the capture subsystem 112 of the capture device 100 may record full resolution video of a subject. The capture device 100 may include a camera configured to generate content at one or more resolutions or other measurements of quality (e.g., video content at one or more of 8K UHD, 4K UHD, 1080p, 720p, etc.; image content at one or more of 200 megapixels, 48 megapixels, 12 megapixels, etc.). The capture subsystem 112 may cause the camera to produce output at a highest resolution available, or at a resolution that is predetermined or dynamically-determined to be the highest resolution to be produced for a particular content item. Such content may be referred to as full resolution content.

In some embodiments, a capture device 100 may have multiple cameras, such as cameras with different lenses (e.g., zoom, wide angle, macro) or cameras facing different directions (e.g., a user facing camera and a rear facing camera). The capture subsystem 112 may activate a particular camera by default, determine which camera is to be used based on the subject and current conditions (e.g., lighting, distance from subject, etc.), or allow a user of the capture device 100 to select which camera is to be used.

At block 604, the capture subsystem 112 may store the full resolution video content in local storage of the capture device 100. For example, full resolution content 222 may be stored in data store 224. In addition, capture subsystem 112 may cause presentation of the full resolution content 222 on a display of the capture device 100.

At block 606, the capture subsystem 112 may generate a version of the full resolution video content for transmission to one or more participant devices 102. In some embodiments, network conditions between the capture device 100 and the participant device(s) 102 may affect transmission of the full resolution video content in a manner that would permit substantially real time presentation by the participant device(s) 102. To provide a live stream of the video content, the capture subsystem 112 may generate a version of the content that is optimized for transmission or otherwise provides an improved live stream experience in comparison with the full resolution video content. For example, the live stream version may be compressed or down-sampled from 8K UHD or 4K UHD to SVGA, VGA, or the like.

FIG. 7 illustrates a capture device 100 capturing, presenting, and storing full resolution video at [A]. At [B], a version of the video optimized or otherwise generated for transmission may be sent to a participant device 102 as a live stream. The participant device 102 may present the live stream content at [C]. The participant device 102 may provide one or more user interface options to obtain the full resolution content from which the live stream version was generated. For example, activation of user interface control 700 may cause a request to be sent to the capture device 100 at [D] to obtain a frame or video portion that is a full resolution version of the live stream version presented on the participant device 102. In some embodiments, user activation of the interface control 700 may cause a request to be sent for a full resolution image of a frame of live video currently being presented or presented immediately preceding activation of the user interface control 700. In some embodiments, user activation of the interface control 700 may cause presentation of a selection interface that allows a user to pause, rewind, and select a frame or video segment for which a full resolution version is to be requested.

At decision block 608, the capture device 100 may determine whether a request has been received from a participant device 102 for a full resolution version of content that has been streamed to the participant device 102. If a request has been received, routine 600 may proceed to block 610. Otherwise, routine 600 may return to block 602 to continue recording full resolution video.

At block 610, the capture device 100 can obtain the requested full resolution content from local storage and send it to the participant device 102. In some embodiments, the request may include a frame identifier or set of frame identifiers that specify a frame or set of frames (e.g., by offset, ID, or timestamp). In some embodiments, the request may include time range data specifying a range of times for a subset of video (e.g., a series of frames of video). Based on the identifying information, the capture device 100 may fetch the requested full resolution content from local storage and provide it to the participant device 102.

To provide the requested full resolution content to the participant device 102, the capture device 100 may open—or use a previously-opened—communication channel that is physically or logically separate from the channel over which the capture device 100 sends live stream content to the participant device 102. In some embodiments, the capture device 100 may send the requested full resolution content via a transmission control protocol/internet protocol (TCP/IP) connection that has been opened for the purpose of communicating separately from the sending of live stream content. In some embodiments, the capture device 100 may use a data transfer session, such as a hypertext transfer protocol (HTTP) or file transfer protocol (FTP) session, to send the full resolution content to the participant device 102. Such a data transfer session may occur over an existing or newly-opened TCP/IP connection. In some embodiments, the capture device 100 may provide the requested full resolution content to an intermediary, such as network-accessible server, from which the participant device 102 can access and download the requested full resolution content. For example, the participant device 102 may access the full resolution content using an application programming interface (API) exposed by the server, a network address provided by the capture device 100, etc. In some embodiments, the capture device 100 may provide the requested full resolution content as an attachment to a message (e.g., an instant messaging chat message as part of a message thread, an electronic mail message, etc.) to the participant device 102. The example communication mechanisms for requesting full resolution content and providing requested full resolution content separately from a live stream content presentation session and/or live bidirectional communication session are provided for purposes of illustration only, and are not intended to be limiting, required, or exhaustive. In some embodiments, additional or alternative mechanisms may be used.

As shown in FIG. 7, the capture device 100 may obtain the requested full resolution video/image from local storage at [E] and provide the requested full resolution video to the participant device 102 at [F]. Advantageously, the capture device 100 may obtain the requested full resolution content from local storage and provide it to the participant device 102 while continuing to capture, present, and store full resolution video. In this way, a request from a participant device 102 for a full resolution content item does not interfere with the continued capture of content as desired by the user of the capture device 100. Thus, the capture device 100 may present different content than the participant device 102 (e.g., as shown at [E], where the capture device 100 continues to capture and present live video content of a subject, and at [G] where the participant device 102 presents requested full resolution content that is not live streamed from the capture device 100) while maintaining a live bidirectional audio connection between the capture device 100 and participant device 102 for audio communication. In some embodiments, the capture device 100 may also continue to send, to the participant device 102 (and in some cases other participant devices 102), live video that is optimized for transmission while fetching and sending the requested full resolution content.

The participant device 102 may store the received content in local storage for playback. For example, if the participant device 102 is a control device 200, the received content may be stored in the data store 260 that is local to the control device 200. Advantageously, receipt and storage of the full resolution video content in local storage of the participant device 102 can permit responsive local control of the full resolution video content.

The participant device 102 may present the received full resolution content at [G]. The user of the participant device 102 may interact with the received full resolution content. For example, the user may activate one or more user interface controls to zoom, rotate, edit, manage playback, create annotations (e.g., snapshots, narrations), move or position a cursor 702, and the like. Metadata regarding the user interactions, edits, additions, and the like may be stored or transmitted to other devices for asynchronous or synchronous presentation. For example, playback metadata may represent playback commands and corresponding timestamps or durations for playback events such as pause, rewind, fast forward, play, zoom/viewport, and the like. As another example annotation metadata may represent coordinates, colors, shapes, and other properties of on-screen markups (e.g., drawn using a stylus or finger) and the timestamps at which they are to be displayed. As a further example, annotation metadata may represent coordinates and content of textual markups, and the timestamps at which they are to be displayed. As an additional example, annotation metadata may represent audio and/or video of the user of the participant device 102, and the timestamps at which they are to be displayed. Generation and consumption of non-destructive annotations are described in greater detail below.

In some embodiments, the user's interactions with the content may be reflected on the display of the capture device 100 or other participant devices in substantially real time. To facilitate synchronization among the devices participating in the live collaboration session, the participant device 102 may transmit the playback metadata, annotation metadata, or both to the other devices participating in the live collaboration session at [H]. In some embodiments, the participant device 102 may also send live audio and live video to the capture device 100, such as video from a user-facing camera of the participant device 102 that may be presented by the capture device 100 (and, in some cases, other participant devices) in an inner window 520 along with the participant device-controlled content, and the like.

At block 612, the capture device 100 may synchronize or otherwise alter display of the capture device 100 based on playback metadata, annotation metadata, or the like received from the participant device 102. For example, the capture device 100 may apply playback events to the full resolution version of content stored in the data store 224 local to the capture device 100 to synchronize the display to that of the participant device 102.

As shown in FIG. 7, the capture device 100 may access a particular frame or portion of content indicated by the playback metadata, and apply a degree of zoom indicated by the playback metadata received from the participant device 102.

In some embodiments, the request by the participant device 102 for full resolution content, the interaction with the full resolution content by the user of the participant device 102, the synchronization of display across devices participating in a live collaboration session, and other interactions may all occur while a bi-directional audio channel remains open. For example, the user of the participant device 102 may discuss aspects of the content being displayed (paused, rewound, played back, zoomed, marked up, etc.), and the live audio may be provided to other devices. Users of other devices may respond audibly and the live audio may be provided to the participant device 102. As another example, a live video feed from a user-facing camera of the participant device 102 may be provided for presentation on the capture device 100/other participant devices in addition to synchronizing display of content to that of the participant device 102.

In some embodiments, multiple participant devices 102 may send interaction data (time stamps, playback commands, annotations, and the like) to each other, enabling substantially simultaneous and bi-directional interactive review of content such as a video segment which was shared among the two devices and is stored in the local storage of both devices, without necessarily involving a capture device 100 (e.g., where the participant devices 102 are not viewing live captured content). Enabling substantially simultaneous and bi-directional interactive review of content, a command sent by any participant device 102 to the other participant device(s) 102 may be simultaneously performed by both/all devices. In some cases, where multiple commands are sent, the commands may be prioritized according to a predetermined order or hierarchy (e.g., having the last command overrule all previous commands, having a command associated with a higher level of a hierarchy overrule a command associated with a lower level, having a command from a device associated with a hither level of a hierarchy overrule a command from a device associated with a lower level). As an example, any participant device 102 may send a “play” command thereby effecting playing of the video content on both/all devices, and thereafter any device may send a “pause” command, effecting pause of the video content at the same frame in both/all devices. Alternatively, one of the participant devices 102 may be associated with a higher level of a control hierarchy than the other participant device(s) 102, and may pause the video such that the other device is not permitted to resume playback due to the other device being associated with a lower level of the hierarchy, or the participant device 102 associated with the higher level of the hierarchy may have the sole ability to erase an annotation, and the like. It is appreciated that the above examples refer to two communicating participant devices 102, but may be generalized and applicable to more than two participant devices 102, thereby allowing group communication. As an example, a capture device 100 or participant device 102 may send the high-quality content as explained above to more than one recipient device simultaneously. As another example, a group of more than two participant devices 102 may review simultaneously a video segment which was previously sent to all group devices by any of the group devices and is stored in the local storage of each of the devices in the group, as explained above. A predetermined command order or hierarchy may be defined for the group-review, for example the last command sent by any participant device 102 will be effected in all participant devices 102, overriding previous commands as applicable. Alternatively, a particular participant device 102 in the group, for example the participant device 102 inviting or initiating the group review or discussion, may be associated with a higher level of the control hierarchy than other participant devices 102 in the group, and as such may have the power to erase annotations made by other devices, pause the video at a certain frame such that other devices cannot resume playback, etc.

### Annotation

FIG. 8 is a flow diagram of an illustrative routine for generating annotated content that may be shared with other devices. Advantageously, the annotations may be non-destructive annotations in the sense that the underlying content (e.g., video content generated by a capture device) is not altered or is otherwise recoverable separately from the applied annotations.

A capture device 100 may generate raw content 800. For example, the capture device 100 may generate raw content 800 by capturing video or still image of a live patient, a screen, a printed image, a document, another subject, or some combination thereof. In some embodiments, the raw content 800 may be generated at the highest resolution or other highest quality setting available to the capture device 100, as described in greater detail above. The raw content 800 may be raw in the sense that any compression, down-sampling, masking, annotations, or other modifications are not reflected in the raw content 800 that is stored by the capture device 100.

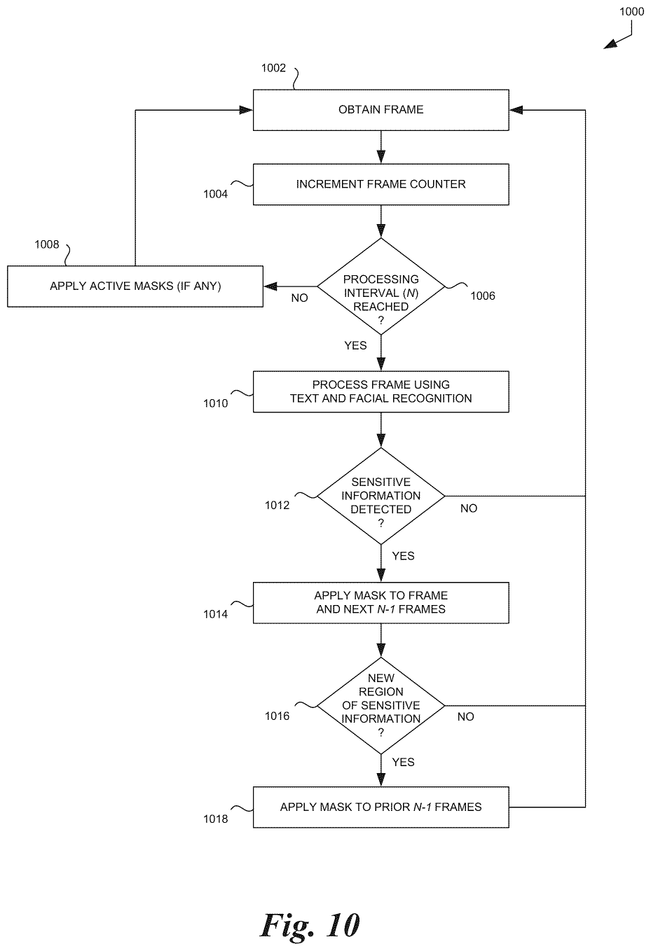

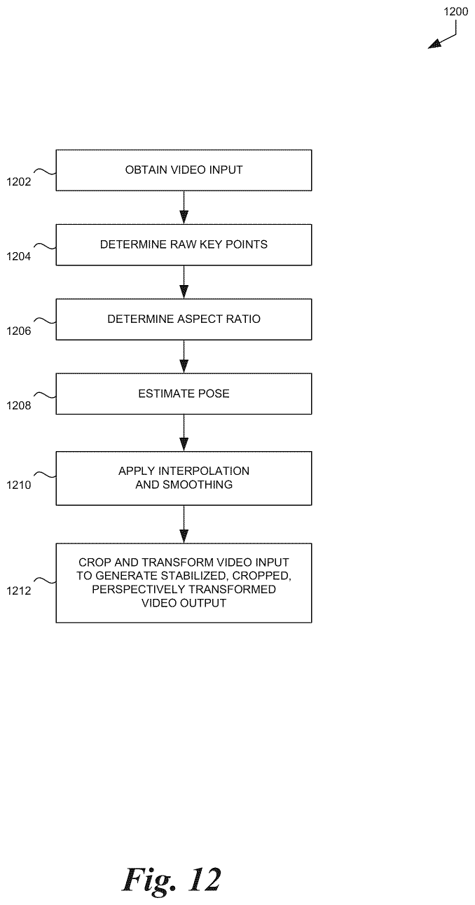

At 810, the capture device 100 may perform augmentation processing on the raw content 800 to generate augmented content that is optimized or altered in some way to improve transmission, presentation, compliance, or the like. In some embodiments, the augmentation processing 810 may include detection and masking of potentially sensitive information, as described in greater detail below with respect to FIG. 10. In some embodiments, the augmentation processing 810 may include stabilization, cropping, and optimized presentation of screen-based content captured in the raw content 800, as described in greater detail below with respect to FIG. 12. In some embodiments, the augmentation processing 810 may include other augmentations, or some combination of augmentations. The augmentations to the raw content 800 to produce augmented content may be stored in an augmentation file 802. For example, the augmentation file 802 may be a file of metadata describing, defining, or referencing modifications to be made to the raw content 800. The augmentation file 802 and raw content 800 together may be used to produce a media file 804. The media file 804 may be a presentable content file, such as a video file, that provides a version of the raw content 800 to which the augmentations defined in the augmentation file 802 have been applied.

In some embodiments, generating the augmentation file 802 may be a multistep or iterative process by which a user applies additional or alternative augmentations to the raw content 800. For example, at 812 the augmented video content may be edited to customize or provide additional or alternative masks for potentially sensitive information, to remove masks from information not potentially sensitive, or to provide additional or alternative cropping or stabilization of content (e.g., screen content, document content, printed image content, or the like).

At 814, a user may apply one or more annotations to the media file 804 to produce one or more annotation files 806. When viewing a media file 804 (e.g., video or image), a user may choose to annotate it by adding overlays such as text, arrows, geometric shapes etc. The user may use the capture device 100, or another device such as a participant device. For example, a participant device may obtain the media file 804 from the capture device by requesting full resolution content from the capture device 100 as described in greater detail above.

In some embodiments, a user may choose to generate the annotation as a snapshot. To generate a snapshot, the device on which the snapshot is being generated may create an annotation file 806 linked to the original media file 804 that can be used to provide an annotated image. The annotation file 806 may include information to reproduce the current view of the media and its overlaid annotation. For example, the annotation file 806 may include or define one or more of the following: an identifier of the image that is to serve as the underlying content for the annotation, an identifier of the current frame in the case that the underlying media file 804 is a video media file (in which case the snapshot functionality is only available when the video is paused); the pan and zoom applied to the original image or video frame (viewport); and all the annotation overlays the user added (e.g., defined as vector graphics).

The media file 804 and annotation file 806 together may be used to present annotated, augmented content. Advantageously, the separation of annotation file 806 from media file 804 may also allow a user to interact with, alter, replace, or remove annotations and interact with the underlying media content defined in the media file 804. This separation of annotations from underlying media may be referred to as non-destructive annotations.

The user of the system may choose to share the annotation file 806 with one or more other users. To provide the other user(s) with the information to present the snapshot, both the annotation file 806 and the underlying media file 804 may be shared. In some embodiments, when opening the annotation file 806, participant device 102 in receipt of the annotation file 806 uses the annotation file 806 and media file 804 to generate a presentation 808 that is a reproduction of the exact snapshot from which the annotation file 806 was generated. For example, if the media file 804 is a video file, the annotation file 806 can indicate a particular timestamp, frame ID, or offset to be presented. Any zoom or pan may be applied to the image. Any annotations may be presented with the image (e.g., as overlays). The user viewing the annotation file can choose to switch from the presentation 808 to the underlying media file 804.

In some embodiments, a user may choose to generate the annotation as a video-based non-destructive annotation, also referred to as a narration. Creating a video-based annotation can include annotating one or more media files (videos, images) and recording a timeline that includes image and video manipulation applied to the selected media file(s) such as: playing, pausing, or performing other playback operations of video; zoom or pan of video or images; modifying image settings such as brightness; recording pointer/cursor movement (e.g., a user-movable arrow-shaped marker) across the video or image; adding annotation overlays such as freehand drawing, geometry shapes, text; and the like. A user creating such a video-based annotation may also switch to another media file, and make any combination of playback settings, modifications, annotation overlays, and the like, on multiple media files, all as part of a single video-based annotation. The process may be repeated as desired.

Annotation metadata defining the video-based annotation may be generated and saved as an annotation file 806. In some embodiments, the annotation file 806 may be a structured file such as a Protocol Buffers (Protobuf) file, JavaScript Object Notation (JSON) file, or Extensible Markup Language (XML) file. The annotation file 806 may define, for each point in time or period of time within the annotation (e.g., n points in time per second, where n is an integer such as 10, 30, 60, etc.), various presentation parameters for the video-based annotation. For example, a particular point in time may include entries for the media file to be presented, the frame to be presented (if the media file is a video), the degree of zoom to be applied, the viewport to be displayed, any vector graphics to be presented, textual markups to be presented, the location at which the vectors graphics or textual markups are to be presented (e.g., pixel locations or other coordinates), other presentation features, or any combination thereof. The annotation file 806 may include entries for each point in time of the video-based annotation. Thus, when the video-based annotation is presented, the presentation features may be presented at each appropriate point in time, thereby providing a dynamic presentation in which the user's actions during recording of the video-based annotation are reproduced on the viewer's screen (e.g., content is dynamically started, stopped, switched, zoomed, panned, marked-up, pointed at, etc.).

While recording a video-based annotation, a user can record a soundtrack of the user's own spoken audio, or mute the device audio recording and create a separate soundtrack to accompany the recording. The audio recording or other soundtrack may be stored in file referenced by the annotation file 806, or the annotation file 806 may include the audio recording or other soundtrack. When generating the video-based annotation is done, the user can playback the annotation locally. The user may send this video-based annotation to one or more other participant devices 102. To send the video-based annotation, the annotation file 806 and underlying media file(s) 804 may be sent. A receiving participant device 102 may generate a presentation 808 using the annotation file 806 to reproduce any video-based annotations, manipulations, and the like over the underlying medial file(s) 804. The user viewing the non-destructive video-based annotated file can choose to switch from the presentation 808 to the underlying media file(s) 804.

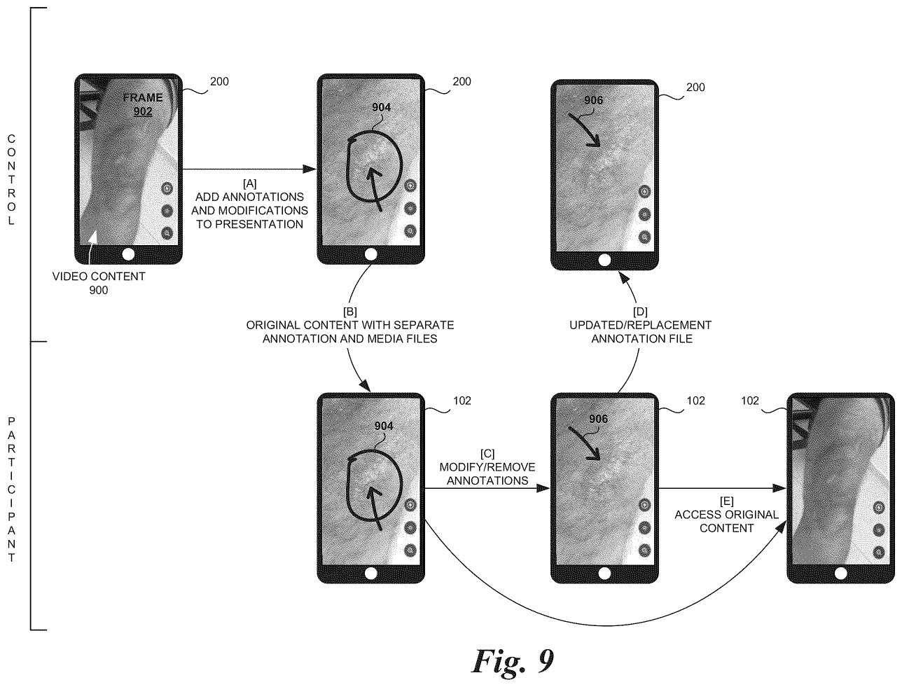

FIG. 9 illustrates an example of a control device 200 generating annotated content for distribution to one or more participant devices 102. At [A], the control device 200 generates annotated content by adding one or more annotations and modifications to presentation of content. For example, the user may have paused video content 900 (either recorded or live capture) on a particular frame 902, zoomed in on a particular portion of the frame 902, and added a drawn annotation 904. Metadata regarding the frame, zoom, and annotation may be generated and saved as an annotation file 806 separate from a media file 804 for the underlying video 900 or frame 902. The control device 200 may send the annotation file 806 and media file 804 to a participant device 102 at [B].

The participant device 102 in receipt of the annotation file 806 and media file 804 may present the annotation by applying, to the content in the media file 804, the annotation features defined in the annotation file 806. Because the media file 804 is provided separately (e.g., logically or physically) from the annotations defined in the annotation file 806, the user may modify or remove annotations at [C]. For example, the user may remove annotation 904 and add a new annotation 906.

In some embodiments, the participant device 102 may provide, to the control device 200 (and other participant devices) at [D], a separate annotation file defining the changed or added annotations made by the user of the participant device 102, and excluding any annotations removed by the user of the participant device 102. The control device 200 (or other participant devices) may then update the presentation of content by applying the annotation file 806 received from the participant device 102 to the previously-received media file 804.

In some embodiments, the user of the participant device 102 may access, at [E], the underlying content to which annotations received from the control device 200. For example, if the media file 804 is a video content item, the user may playback the unannotated video.

### Masking of Sensitive Information

FIG. 10 is a flow diagram of an illustrative routine 1000 for masking potentially sensitive information in video content. Potentially sensitive information may include, but is not limited to, personally-identifiable data, health data, human faces, and the like. Advantageously, a capture device 100 may perform the routine 1000 or portions thereof to automatically mask sensitive information that is present on a screen, image, document, or otherwise captured by a camera of the capture device 100. Thus, the video content may be shared with one or more participant devices 102 without exposing the sensitive information. Such video content may be referred to as anonymized video content.

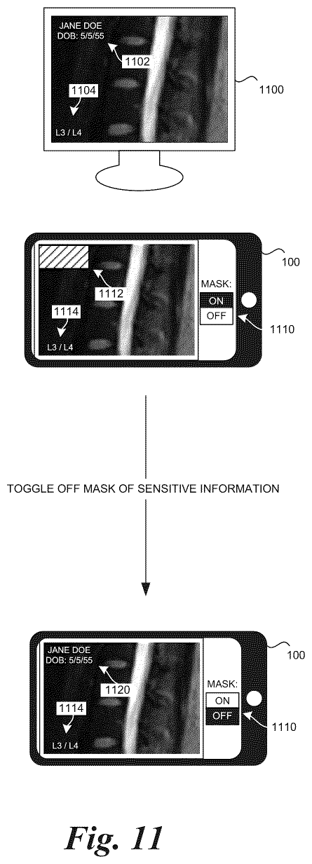

Portions of FIG. 10 will be described with further reference to the example screen and video content thereof illustrated in FIG. 11. Although the example shown in FIG. 11 and described below is a screen, the example is provided for purposes of illustration only and is not intended to be limiting or required. In some embodiments or in some situations, video content may include video of a printed image or document, a live patient, or some other source of potentially-sensitive information. The techniques for detecting potentially-sensitive information and masking it in video content may apply to these print-out and live patient situations and any other source of potentially-sensitive information that may be captured in video content.

The routine 1000 may be computer-implemented method that begins in response to an event, such as when the capture device 100 begins capturing video content, or when a device initiates playback, editing or review of an already-stored video content which includes potentially-sensitive information. When the routine 1000 is initiated, a set of executable program instructions stored on one or more non-transitory computer-readable media (e.g., hard drive, flash memory, removable media, etc.) may be loaded into memory (e.g., random access memory or “RAM”) of a computing device, such as the computing device 1700 shown in FIG. 17 and described in greater detail below. In some embodiments, the routine 1000 or portions thereof may be implemented on multiple processors, serially or in parallel.

At block 1002, the capture subsystem 112 of the capture device 100 may obtain a frame of video from the camera of the capture device 100. The video frame may be generated in the highest resolution that the camera of the capture device 100 is configured to generate (e.g., 8K UHD, 4K UHD, 1080p, etc.). In the description that follows, in order to distinguish (1) the most-recently captured or generated frame that triggered or otherwise immediately preceded execution of the current iteration of routine 1000, from (2) previously-received or generated frames, the most-recently captured or generated frame will be referred to as the “current” frame, and the time at which the current input data was captured or generated may be referred to as the “current” time. Accordingly, previously-captured or generated frames will be referred to as “prior” frames, and the time at which a prior frame was captured or generated may be rereferred as a “prior” time.

At block 1004, the capture subsystem 112 may increment a frame counter. In some embodiments, the frame counter may be used to determine when to review frames for the presence of potentially sensitive information. For example, the capture subsystem 112 may only review every Nth frame in detail (where N is a predetermined or dynamically-determined value), rather than reviewing each and every frame. By reviewing only every Nth frame (e.g., about every 5th frame, about every 10th frame, about every 30th frame, about every 60th frame, or about every 120th frame), the processing requirements in terms of power consumption, processor usage, and the like may be reduced.

At decision block 1006, the capture subsystem 112 may determine whether a processing interval has been reached. For example, the capture subsystem 112 may compare the current frame counter value to the processing interval N. If the frame counter value equals or exceeds the processing interval, the routine 1000 may proceed to block 1010 for processing of the frame. Otherwise, the processing interval has not been reached, the routine 1000 may proceed to block 1008.

At block 1008, the capture subsystem 112 may apply previously-determined, active masks to regions of potentially sensitive information in the current frame, as described in greater detail below.

At block 1010, the capture subsystem 112 can process the current frame to detect regions (if any) in which potentially sensitive information may be present. The processing may include one or more preprocessing steps to identify candidate information.

In some embodiments, capture subsystem 112 may perform optical character recognition (OCR) to identify textual content in the current frame. Once textual content is detected and extracted in textual form (e.g., as a character string rather than pixels of the current frame), the textual content may be evaluated to determine whether it is potentially sensitive information. For example, a machine learning model may be trained to classify text as being potentially sensitive information, or not potentially sensitive information. The machine learning model may be a neural network, transformer, support vector machine, Bayesian network, or some other machine learning model that may be trained to classify textual input. A set of training data may be obtained or generated, including a first subset of sensitive information and a second subset of information that should not be classified as sensitive information. The training data may be used to train the model. The specifics of training the model depend on the type of model (neural network, transformer, etc.). Once the model is trained, it may be deployed to capture devices 100 for use in classifying textual content.

In some embodiments, capture subsystem 112 may perform object recognition to identify potentially sensitive visual information in the current frame. For example, a machine learning model may be trained to perform facial recognition to identify faces or portions thereof in frames of video content. The machine learning model may be a convolutional neural network (CNN), You Only Look Once (YOLO) model, or some other machine learning model that may be trained to perform facial recognition or other object recognition. A set of training data may be obtained or generated, including a first subset of sensitive visual information and a second subset of visual content that should not be classified as sensitive visual information. The training data may be used to train the model. The specifics of training the model depend on the type of model (CNN, YOLO model, etc.). Once the model is trained, it may be deployed to capture devices 100 for use in classifying visual content.

At decision block 1012, the capture subsystem 112 can determine whether any regions of the current frame include potentially sensitive information. If so, the routine 1000 may proceed to block 1014. Otherwise, if no regions of potentially sensitive content have been detected in the current frame, the routine 1000 may return to block 1002 to perform a subsequent iteration on a next frame (if any).

FIG. 11 illustrates an example screen 1100 displaying visual content. In this example, the visual content is an MRI image of a portion of a patient's spine. The display on the screen 1100 also includes two regions of text: region 1102 which identifies the patient's name and birthday, and region 1104 which identifies the portion of spine displayed in the MRI image. A capture device 100 may capture video of the screen 1100, and a capture subsystem 112 of the capture device may process frames of video content to detect potentially sensitive information.

In the illustrated example, the capture subsystem 112 may have performed OCR to identify the two text regions and determine the text displayed therein. The capture subsystem 112 may then have evaluated the text to determine whether it is potentially sensitive information (e.g., using a machine learning model trained to classify textual content as including or not including potentially sensitive information). The text in region 1102 may have been classified as including potentially sensitive information due to the inclusion of the patient's name and birthday, while the text in region 1104 may have been classified as not including potentially sensitive information because the text consists of medical terms describing what is depicted in the image. The capture subsystem 112 may also have evaluated the non-textual visual content displayed on the screen 1100 to determine whether any potentially sensitive information that is not textual has been displayed. In this example, a facial recognition model may have been used to determine that there are no regions in the view of the screen 1100 that include faces or facial features (e.g., eyes) that would be personally identifiable and therefore potentially sensitive.

Returning to FIG. 10, at block 1014 the capture subsystem 112 can implement one or more measures to prevent dissemination of potentially sensitive information detected in the current frame. In some embodiments, the capture subsystem 112 can apply one or more masks to the current frame. Application of masks may include blurring one or more regions (e.g., via pixelation), applying a visual overlay (e.g., an area of blackout or whiteout), or implementing some other technique to block a view of a portion of the current frame.

In some embodiments, in addition to applying a mask to any regions of potentially sensitive information in the current frame, the capture subsystem 112 may store active mask data regarding the regions of potentially sensitive information for use in applying masks to the frames that will be captured before the next processing interval is reached. For example, the active mask data may be used at block 1008 to apply masks to the next N−1 frames (where N is the processing interval as described above), without processing the frames through blocks 1010-1018. Advantageously, application of a mask to subsequent frames can ensure masking of any potentially sensitive information that continues to appear for one or more frames after the current frame but before the next frame for which the processing interval is reached.

In the example shown in FIG. 11, the display of the capture device 100 shows video content of screen 1100 being captured (or previously captured). A mask has been applied to the region 1112 of the video content displayed on capture device 100 that corresponds to region 1102 of the screen 1100 because region 1102 was previously determined to include potentially sensitive information. The mask is indicated by diagonal lines in the illustrated example; in practice, the mask may be any other visual effect that blocks viewing of the potentially sensitive information. No mask has been applied to the region 1114 of the video content displayed on capture device 100 that corresponds to region 1104 of the screen 1100 because region 1104 was previously determined to not include potentially-sensitive information.

At decision block 1016, the capture subsystem 112 can determine whether any region of potentially sensitive information that has been detected in the current frame is a new region of potentially sensitive information. In this context, a “new” region of potentially sensitive information may be a region of content detected in the current frame that was not detected in the last frame that was analyzed (e.g., if the current frame is frame i, then a new region would be a region of potentially sensitive information that was not detected in frame i−N, which was the last frame for which the processing interval was reached). The identification of a new region of potentially sensitive information compared to a prior frame for which the processing interval was reached may be based on one or more identifying factors.

In some embodiments, a region of potentially sensitive information may be defined in terms of location within the frame. For example, coordinates such as those based on pixels or other coordinate systems may be used to identify the location and size of a region of potentially sensitive information. The coordinates may form a bounding region, such as a rectangle or other polygon. The locations of regions of potentially sensitive information identified in the current frame may be compared with the location of regions of potentially sensitive information identified in the prior frame. If a region identified in the current frame has the same location or substantially similar location (e.g., within a threshold coordinate value) as a region in the prior frame, then the region in the current frame may not be considered a new region of potentially sensitive information. If a region identified in the current frame does not have the same or substantially similar location as any region in the prior frame, then the current frame may be considered a new region of potentially sensitive information.

In some embodiments, a region of potentially sensitive information may be defined in terms of the content of the region. For example, textual content or pixel content may be used to identify the location and size of a region of potentially sensitive information. The textual or pixel content of regions of potentially sensitive information identified in the current frame may be compared with the textual or pixel content of regions of potentially sensitive information identified in the prior frame. If a region identified in the current frame has the same textual or pixel content or substantially similar textual or pixel content (e.g., within a threshold comparison metric) as a region in the prior frame, then the region in the current frame may not be considered a new region of potentially sensitive information. If a region identified in the current frame does not have the same or substantially similar textual or pixel content as any region in the prior frame, then the current frame may be considered a new region of potentially sensitive information.

At block 1018, if a new region of potentially sensitive information has been identified in the current frame, the capture subsystem 112 may apply a mask to one or more prior frames of video content. Advantageously, application of a mask to prior frames when a new region of potentially sensitive information is detected can ensure masking of any potentially sensitive information appearing for the first time before the current frame but after the previous frame for which the processing interval was reached.

In some embodiments, the determination of which frames or how many frames may be based on the processing interval (e.g., N as described above). For example, if the video content is being provided to participant devices as a substantially live stream, then the capture subsystem 112 may maintain a buffer of at least N−1 frames. If a new region of potentially sensitive content has been identified, then a mask may be applied to the same region (e.g., the same coordinate locations) in each of the prior N−1 frames in the buffer.

Although FIG. 10 illustrates routine 1000 as being implemented such that not every frame is evaluated for potentially sensitive information, the illustration is provided for purposes of example only and is not intended to be limiting or required. In some embodiments, every frame of video content may be individually evaluated for potentially sensitive information. For example, blocks 1004, 1006, 1008, 1016, and 1018 may not be performed, and the operations of block 1014 may be limited to applying a mask to only the current frame.

With reference to FIG. 11, in some embodiments a selectable option may be provided to allow the user of the capture device 100 to control whether masks are displayed. As shown, an interactive mask control 1110 such as a toggle may be provided. Activation of the mask control 1110 may cause presentation of masks determined in routine 1000, while deactivation of the mask control 1110 may remove the masks and allow presentation of underlying sensitive information on the capture device 100. For example, deactivation of the mask control 1110 may remove mask region 1112 and uncover text region 1120, which corresponds to region 1102 of screen 1100.

In some embodiments, toggling the mask control 1110 does not affect the underlying video content. The capture device 100 may generate and locally store an unmasked version of the video content. Mask data regarding masks to be displayed with the video content may be stored separately but in connection with the video content. For example, mask data may include coordinates such as those based on pixels or other coordinate systems to identify the location and size of each region of potentially sensitive information to be masked. The coordinates may form a bounding region, such as a rectangle or other polygon. The mask data for each mask may further include frame identifiers for the frame or frames to which the mask defined by the mask data is to be applied. By storing mask data rather than applying masks to video content for storage, the toggle functionality illustrated in FIG. 11 is enabled for both live video content and stored video content that is played back. In some embodiments, to ensure that regions of potentially sensitive information are not exposed to participant devices, the masks defined by mask data may be applied to the video content that is sent to the participant devices instead of providing the mask data separately from the unmasked video content.

In some embodiments, users may add or remove individual masks. User interaction data may be generated representing a user interaction with a region of the display of the capture device 100, and the capture subsystem 112 may toggle display of a mask on the region. For example, a user may tap or otherwise select mask region 1112 to remove the mask and cause display of underlying text region 1120. As another example, a user may tap, circle, or otherwise select region 1114 (or another region) and cause presentation of a mask over the indicated region. The mask data associated with the video content may be updated accordingly (e.g., by removal or addition of mask data, as needed).

In some embodiments, user interactions to add or remove individual masks may be used to train or update one or more models used to identify potentially sensitive information. When a user removes a mask that has been added based on detection of potentially-sensitive data, a frame or frames with underlying content exposed by the user may be saved and used in a subsequent training process (e.g., the portion of the frame(s) exposed by the user may be labeled as negative for presence of potentially sensitive information). When a user adds a mask to a region of a frame that has not been masked and therefore that has not been previously determined as including potentially sensitive information, a frame or frames with underlying content masked by the user may be saved and used in a subsequent training process (e.g., the portion of the frame(s) masked by the user may be labeled as positive for presence of potentially sensitive information).

### Digital Stabilization of Information-Rich Content

FIG. 12 is a flow diagram of an illustrative routine 1200 for stabilizing the presentation of information-rich objects, such as documents, printed images, or display screens. A capture device 100 (or an external video transformation system) may perform the routine 1200 or portions thereof to automatically smooth the effect of camera motion and flatten the perspective of generally rectangular information-rich objects. Such smoothing and perspective transformation can be particularly advantageous when the capture device 100 is a handheld device, such as a smart phone or tablet or a portable digital camera, and the information-rich object is a substantially stationary object. Moreover, a smoothed, perspective-transformed view of such an object can be cropped to provide a full-frame or substantially full-screen view of the object to further improve the view provided to recipients.

In the description that follows, the operations are described as being performed by a capture subsystem 112 of a capture device 100, and the input is described as being video input data. However, the description is provided for purposes of illustration only, and is not intended to be limiting. In some embodiments, the operations (or a subset thereof) may be performed by an external video transformation system. In some embodiments, the routine 1200 may be used to crop and transform the perspective of still images instead of, or in addition to, video.

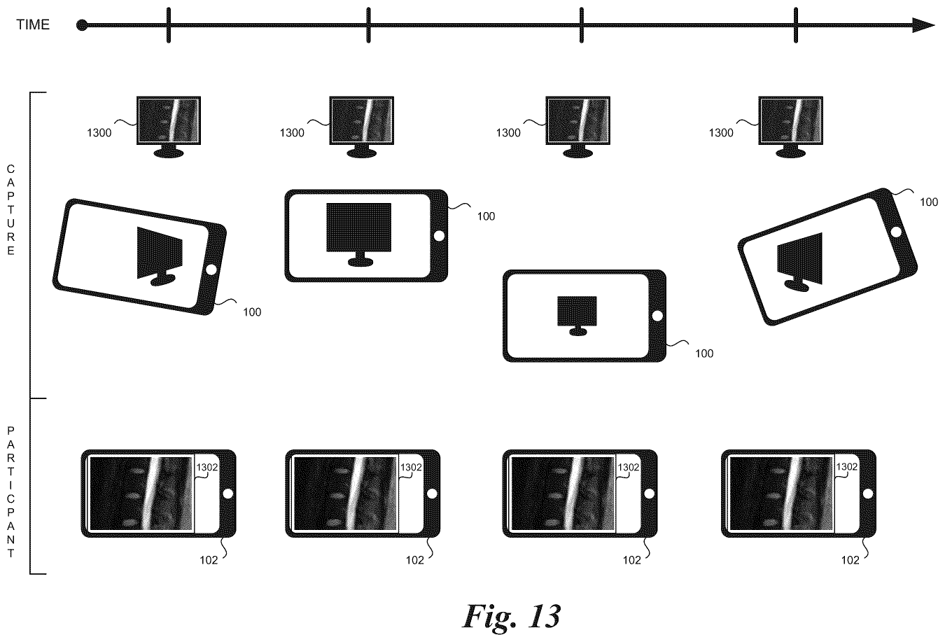

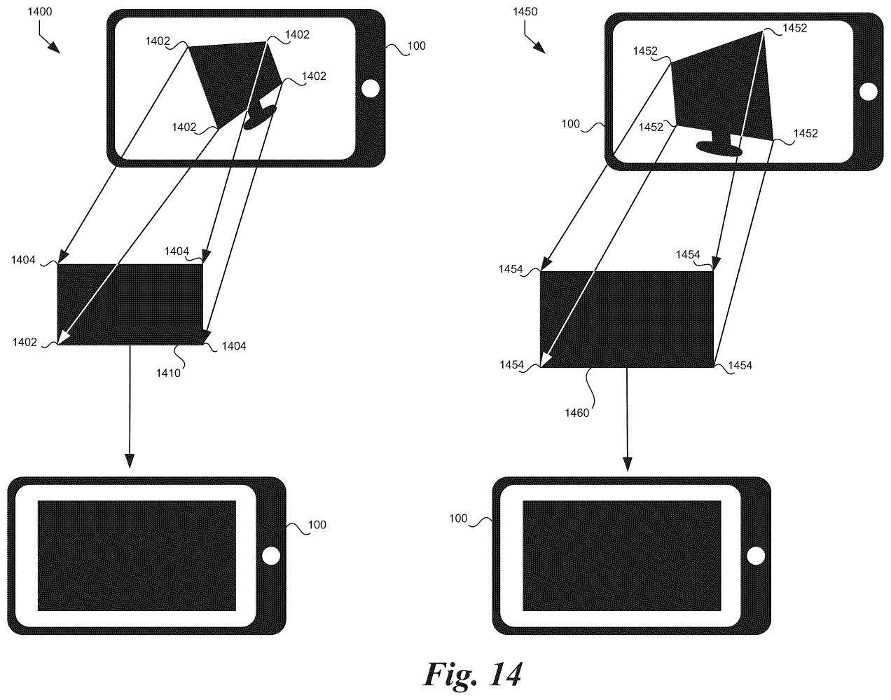

Portions of FIG. 12 will be described with further reference to the example screen and video capture thereof illustrated in FIGS. 13 and 14. Although the examples shown in FIGS. 13 and 14 and described below focuses on a display screen as the information-rich object that is the subject of video content, the example is provided for purposes of illustration only and is not intended to be limiting. In some embodiments or in some situations, the subject of the captured content may be or include a printed document, image (e.g., a medical scan), or some other object.

FIG. 13 shows a screen 1300 displaying a medical image, and a handheld capture device 100 is capturing video of the screen 1300. As shown, over the course of time the capture device 100 is moving such that the screen 1300 captured in the image and shown on the capture device 100 is moving. For example, a user holding a capture device 100 may purposefully or inadvertently move during capture of video, thereby causing shaking, video capture at varying angles, perspective distortion, and the like. Due to the motion of the capture device 100, it can be difficult to make out sufficient detail in the screen 1300.

To stabilize, crop, and/or transform the view of the screen 1300, the capture device 100 (or an external video transformation system) may utilize pose assessment, three-dimensional geometric perception of properties of a solid object, and/or properties of hand movements (e.g., reasonable hand movements) to perform various operations. For example, the capture device 100 may extract a display surface of the object—also referred to as a “face”—and apply a perspective transformation to produce a cropped, transformed-perspective video that is stable from a particular viewpoint (e.g., a viewpoint perpendicular to the face). To do so, key points (e.g., the corners of the face) may be determined in order to establish a rectangle aspect ratio of the face. Additionally, or alternatively, information on a three-dimensional pose of the object, relative size of the object, and/or three-dimensional location of the object may be determined. Denoising filtering may be applied to the data (e.g., to the object pose rotation angles, object location, and object size changes) to reconstruct the location of the key points. Interpolation may be used to create key points where data is determined to be an outlier or is missing. The denoised, filtered, interpolated set of key points may then be used to generate a smooth, stabilized cropping and perspective transformation of the video. Advantageously, in some embodiments the processing may be performed without motion data from a motion sensor of the capture device 100; rather, it may be based on analysis and processing of the video itself. The routine 1200 shown in FIG. 12 illustrates one embodiment of such processing.

The routine 1200 may be computer-implemented method that begins in response to an event, such as when the capture device 100 begins capturing video content, when an external video transformation system received video content, or when a device initiates playback, editing or review of an already-stored video content. When the routine 1200 is initiated, a set of executable program instructions stored on one or more non-transitory computer-readable media (e.g., hard drive, flash memory, removable media, etc.) may be loaded into memory (e.g., random access memory or “RAM”) of a computing device. In some embodiments, the routine 1200 or portions thereof may be implemented on multiple processors, serially or in parallel.

At block 1202, the capture subsystem 112 of the capture device 100 may obtain video input data from the camera of the capture device 100. The object in the field of view of the camera of the capture device 100, and therefore the subject of the video input data, may be an information-rich object. For example, the subject may be a still image of scan (e.g., x-ray, MRT, CT), a video from an arthroscope or endoscope, textual medical information, other medical information, or some combination thereof. A user may use a handheld capture device 100 to capture a video of the object. The user may purposefully or inadvertently capture portions of the display environment (e.g., other people, environmental objects, etc.) in addition to the object. The user may purposefully or inadvertently move the handheld user device during recording, causing undesirable shaking of the captured video. Additionally, or alternatively, the user may purposefully or inadvertently capture at least a portion of the video from an angle, causing a perspective view of the display screen in the captured video.

In some embodiments, prior to proceeding to subsequent blocks or after executing one or more subsequent blocks of routine 1200, the capture subsystem 112 may adjust one or more capture parameters of the capture device 100 or apply one or more post-capture filters or other processing to optimize the video or still image input data based on the object of interest. Capture parameters may be tuned or post-capture processing may be performed based on the object of interest itself within the frames of video or still image input data rather than on the entire frame or on predetermined general parameters.

For example, if the object of interest is a screen, then once the screen's quadrilateral is identified (e.g., using the method described below), the capture subsystem 112 may adjust the focus of the camera to the center of the quadrilateral, change the exposure level (and resulting brightness of the image) based on the measured light intensity within the detected quadrilateral, etc. This is advantageous for getting an optimized image of the object of interest, disregarding the optical characteristics of the background and surrounding environment, such as when capturing a screen or monitor displaying a surgical operation in a dark operating room.

As another example, if the object is an organ or a lesion or pathology on the surface of an organ, such as a rash, a skin lesion, a scar, a bleeding tissue, the capture parameters may be adjusted to enhance or balance the visual characteristics of that object. If the object is a skin rash or a bleeding, the capture device may detect and analyze the amount of red within the area of interest (e.g., the area in the frame occupied by the object), and employ a corresponding red enhancement digital filter to optimize the view of “reddish” features of the object. If the object is a suspected skin cancer or other dark gray lesion, the capture subsystem 112 may sample the brightness separately inside and outside the borders of the lesion, and utilize this sampled data to optimize the view of the lesion in the captured image/video, such as by employing two different sets of parameters for the brightness level inside and outside the lesion's borders.

At block 1204 the capture subsystem 112 can determine raw key points in the video input data. In some embodiments, the capture subsystem 112 system can perform coordinate identification for the corners of a rectangular shape of the object that is the subject of the video content (e.g., a display screen or other rectangular object). For example, the capture subsystem 112 may implement a model (e.g., a pre-trained computer vision model) configured to detect the location of the corners. Various models may be used; in one example, the model may utilize CenterNet for the initial detection of the key points of an object and a Space-Time Correspondence Network (STCN) for key point tracking. It will be understood that the model may be any other key point/semantic segmentation detection model. In some embodiments, the capture subsystem 112 can perform coordinate identification for any object identified in the video input data, rather than a single rectangular object.

The capture subsystem 112 can perform the coordinate identification in real-time as video input data is received, or in batches after the video input data is received. The capture subsystem 112 can identify a set of key points in all or a portion of the frames (e.g., every n frames, where n can be any number) using computer vision algorithms. The set of key points can serve as detected points of the edges of a rectangle. For example, the set of key points may be vertices forming a quadrilateral. The capture subsystem 112 may utilize the set of key points as identified coordinates in the steps below. Such coordinates may be referred to as “raw frame coordinates.”

FIG. 14 illustrates an example of determining raw frame coordinates for two frames of video data: frame 1400 and frame 1450. In frame 1400, the capture subsystem 112 identifies raw frame coordinates 1402, corresponding to the corners of the screen shown on the display of capture device 100. In frame 1450, the capture subsystem 112 identifies raw frame coordinates 1452, corresponding to the corners of the screen shown on the display of capture device 100. Although the screens shown in each of the frames 1400, 1450 are captured by the capture device 100 at different angles and distances, the capture subsystem 112 may nevertheless identify raw frame coordinates 1402, 1452 using the process described above.

At block 1206, the capture subsystem 112 can detect an aspect ratio of the object in the video input data using the raw frame coordinates. In some embodiments, the capture subsystem 112 may access or be programmed with a list of common or known rectangle aspect ratios defining the ratio between the width and the height of the rectangle vertices (e.g., common aspect ratios of display screens, printed images, and the like). Using the raw frame coordinates and the list of known aspect ratios, the capture subsystem 112 may determine the most likely aspect ratio for the object in the video input data.

In some embodiments, the capture subsystem 112 may perform the following process for each known aspect ratio:
1. Construct a set of three-dimensional coordinates (e.g., corners) for a reference object with a flat surface having a ratio of width to height that is equal to the current aspect ratio being processed.
2. Find a three-dimensional pose of the reference object (e.g., using Solve Perspective-n-Point) matched to the raw frame coordinates previously determined for the object in the video input data. The three-dimensional pose may be defined in terms of rotation and translation vectors.
3. Using the rotation and translation vectors determined above and the raw frame coordinates, project the object in the video input data into two dimensions using 3D-to-2D projection to obtain set of coordinates representing the object (e.g., the corners of the object) in two-dimensional space.
4. Measure the compound distance between the raw frame coordinates and the projected coordinates in two-dimensional space determined above.

Once the process described above has been performed for each known aspect ratio (or a subset thereof), the capture subsystem 112 can determine the aspect ratio with the smallest compound distance. The aspect ratio with the smallest compound distance may be identified as the aspect ratio of the object in the video input data, and may be referred to as the “detected aspect ratio.” To confirm that the detected aspect ratio is the proper or “best fit” aspect ratio for the object in the video input data, the process described above may be performed using raw frame coordinates determined from one or more other frames of video input data.

At block 1208, the capture subsystem 112 can estimate a three-dimensional pose of the object in the video input data. In some embodiments, the capture subsystem 112 may use the detected aspect ratio and a 3D pose estimation method (e.g., Solve Perspective-n-Point, a P3P algorithm, an EPnP algorithm, a SQPnP algorithm, or any other solve perspective algorithm) to determine rotation and translation vectors for the object in the video input data. For example, the capture subsystem 112 may determine rotation and translation vectors for every video frame for which raw frame coordinates have been determined. The resulting vectors may be referred to as “frame pose vectors.” The frame pose vectors can be used to generate, based on 3D-to-2D projection and the reference object, a set of new set of points on the video frame that may be referred to as “frame pose coordinates.” For example, a set of frame pose coordinates may be determined for each frame of video input data, or a subset thereof.

FIG. 14 illustrates an example of determining frame pose coordinates for two frames of video data: frame 1400 and frame 1450. For frame 1400, the capture subsystem 112 generates frame pose coordinates 1404, corresponding to the raw frame coordinates 1402. For frame 1450, the capture subsystem 112 identifies generates frame pose coordinates 1454, corresponding to the raw frame coordinates 1452. Although the screens shown in each of the frames 1400, 1450 are captured by the capture device 100 at different angles and distances, the capture subsystem 112 may nevertheless identify frame pose coordinates for a transformed-perspective version 1410 and 1460 of the screen for frames 1400 and 1450, respectively.

In some embodiments, the capture subsystem 112 can measure the quality of the raw frame coordinates by calculating the aggregated distance between the raw frame coordinates and the corresponding frame pose coordinates determined based thereon. If the distance is greater than a predetermined threshold, that can be an indicator that the raw frame coordinates do not conform with the detected aspect ratio. In response, the capture subsystem 112 may mark the raw frame coordinates as invalid and exclude results determined based on invalid raw frame coordinates.

At block 1210, the capture subsystem 112 can apply interpolation and smoothing to the video input data based on the frame pose coordinates in order to produce data that may be used to generate a view of the object in the video input data that does not move around or transform on a perspective basis.

In some embodiments the capture subsystem 112 can apply a linear interpolation algorithm to augment data for video frames where there is no detection or invalid detection of raw frame coordinates or frame pose coordinates. For example, the capture subsystem 112 may interpolate in a linear fashion the values of the various rotation and translation vectors. In some embodiments, the system can apply a smoothing and denoising algorithm on every value of the rotation and translation vectors independently throughout the video to smooth and denoise the rotation and translation of the object in the video input data being tracked. Examples of smoothing and denoising algorithms that may be used include, but are not limited to, a Savitzky-Golay filter and a One Euro Filter.

At block 1212, the capture subsystem 112 can crop and transform video input data to generate stabilized, cropped, perspective-transformed video output. In some embodiments, the capture subsystem 112 can use the interpolated, smoothed, denoised rotation and translation vector values generated above to compute a set of two-dimensional points for each frame. For example, the capture subsystem 112 may use a reference object with the detected aspect ratio, intrinsic properties of the camera, the rotation and translation vector values, and a point projection method to compute a set of two-dimensional points. One example of a point projection method that may be used is a pinhole camera model that takes into consideration certain intrinsic properties of the camera of the capture device 100, such as focal length, to establish the two-dimensional coordinates in the camera image given the three-dimensional position and the rotation and translation vectors. The set of two-dimensional points for each frame may be referred to as “smooth frame coordinates.” The smooth frame coordinates for a given frame form a quadrilateral that provides a two-dimensional rectangular outline of the object (e.g., the screen, document, or printed image) in the frame. However, the outline may not be consistently oriented and sized from frame-to-frame. For example, the outline may appear rotated from one frame to the next or otherwise not oriented in an expected manner. As another example, the outlines may appear to be sized differently from one frame to the next, even though they conform to the same aspect ratio. To address these issues, the capture subsystem 112 may use affine transformation image processing to crop, rotate and scale the quadrilateral to a new video frame with a fixed width and height that continues to adhere to the detected aspect ratio. This process may be performed for each frame of video to be output.

Although FIG. 12 and the description above set forth a particular multi-step process in which each step builds on the result of a prior step, the example is provided for purposes of illustration only and is not intended to be limiting or required. In some embodiments, some blocks of routine 1200 may be omitted or preformed in an alternative manner. For example, a capture subsystem 112 may determine an orientation of a generally rectangular object in three-dimensional space, and generate a two-dimensional substantially full-screen or full-frame representation of the generally rectangular object based on the determined orientation of the generally rectangular object. In this example, the capture subsystem 112 does not determine an aspect ratio of the generally rectangular object as part of the process.

FIG. 13 illustrates an example of the output of routine 1200. As shown, the view 1302 of the screen 1300 on the participant device 102 after processing is cropped, rotated, and resized to be substantially full frame and perspective-transformed in a consistent manner from frame to frame over the entire time line of the video content, even though the capture device 100 is moving and reorienting with respect to the screen 1300.

### Chat, Discussion, and Case Management

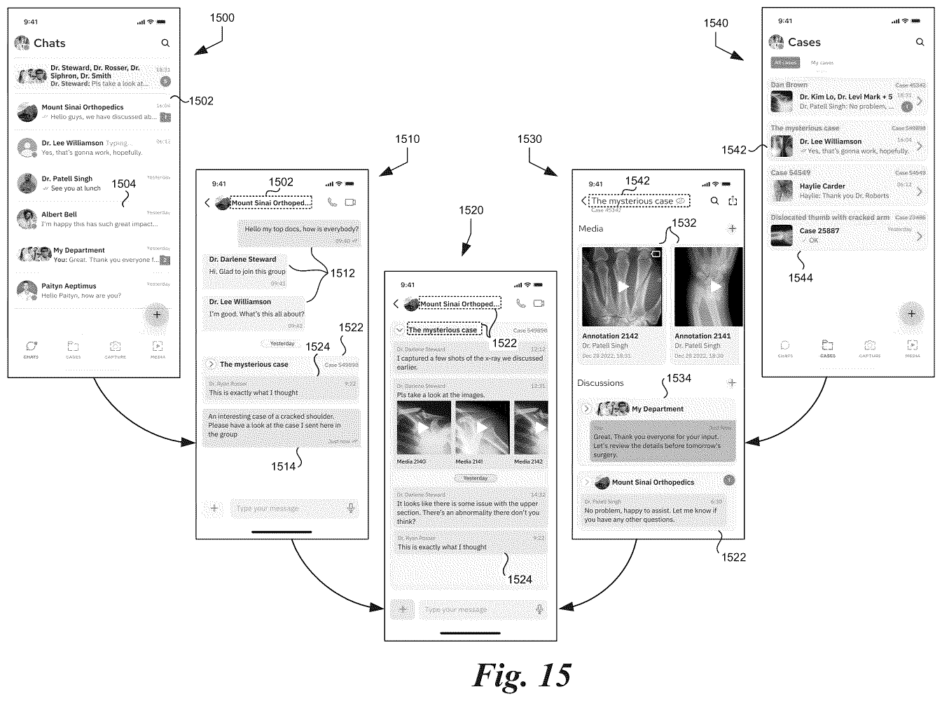

FIG. 15 illustrates example user interfaces of a collaboration system that organizes chat communications, case files, and discussions. The interfaces may be generated and presented on participant devices 102 (including capture devices 100 and/or control devices 200), for example by the conference and chat subsystem 110 shown in FIG. 1. The collaboration system may be wholly or primarily a peer-to-peer system such that messages and other content sent by one device to other devices are stored locally on each device participating in a particular conversation or discussion. In some embodiments, the collaboration system may be managed by a server-based computing system, such as a server of the communication and chat platform 240 shown in FIG. 2. For example, the messages and other content sent by one device to other devices may be stored in and accessed via the communication and chat platform 240.

Generally described, users may communicate with other users in chat conversations (also referred to as “chats” for brevity) that are not necessarily directed to any particular topic. In this context, a chat may be defined in terms of the set of users participating. The chat may include a thread of messages (e.g., text-based messages) that are accessible to the users participating in the chat. In some embodiments, participants may include files as attachments to the messages in the chat. For example, images, videos documents, or other content (e.g., snapshots and narrations generated as described in greater detail above) may be attached to messages or otherwise included in a chat.

Alternatively, or in addition, users may communicate with other users and share content in case discussions (also referred to as “discussions” for brevity) that are directed to a particular topic (also referred to as a “case”). In this context, a discussion may be defined in terms of the particular case being discussed (e.g., a particular medical case). The discussion may include a thread of messages (e.g., text-based messages) that are accessible to the users participating in the discussion. Participants may attach files to a case or discussion. For example, snapshots and narrations generated as described in greater detail above may be attached to cases and discussions for review or discussion.

Advantageously, the collaboration system may manage the assignment of particular chats (e.g., particular threads of chat messages) to particular cases in a manner that maintains separation among each chat and maintains separation among each case, while facilitating discussion of cases in chat conversations and facilitating access to chat conversations alongside case files and other attachments. Thus, a complete conversation history among a particular group of users may be preserved separately from a complete history of a particular case, while allowing users to switch back and forth between chat-centric and case-centric views of information related to both a chat and a case. For example, it can be important to maintain the history, chronology and completeness of data (messages, attachments) related to a medical case so that users can refer back to any symptoms reported, medications prescribed, recommendations made, referrals given, and follow-ups conducted.

As shown in FIG. 15, a chat listing interface 1500 presented on a particular participant device 102 organizes chats by the participating users. Chat 1502 includes a particular group of users (the “Mount Sinai Orthopedics” user group in this example), while chat 1504 includes only one other participant (“Albert Bell” in this example). The user may select a particular chat, such as chat 1502, and access the messages that are part of the chat in a chat interface 1510. In the illustrated example, the user has selected chat 1502, and chat interface 1510 presents chat messages 1512 that are part of chat 1502.

In some embodiments, the chat 1502 and the chat messages 1512 that are part of the chat 1502 are only accessible to the participants in the chat 1502, rather than being accessible to all users of the collaboration system. For example, if Albert Bell is not a member of the Mount Sinai Orthopedics group, then Albert Bell would not be able to access chat 1502 or any of corresponding chat messages 1512. Indeed, chat 1502 would not be shown in the chat listing interface 1500 presented to Albert Bell. Similarly, any messages that are part of chat 1504 are only accessible to the current user and to Albert Bell, even if Albert Bell is part of the Mount Sinai Orthopedics group and a participant in chat 1502.

A case listing interface 1540 presented on a particular participant device 102 organizes content by the case rather than by the participating users accessing the case content. Case 1542 includes content regarding, attached to, or otherwise associated with a particular case (“The mysterious case”), while case 1544 includes only content regarding, attached to, or otherwise associated with a different case (“Dislocated thumb with cracked arm”). The user may select a particular case, such as case 1542, and access the content that is part of the case in a case interface 1530. In the illustrated example, the user has selected case 1542, and case interface 1530 presents content that is part of case 1542. For example, the content includes attachments 1532, which may be annotations (e.g., snapshots, video-based annotations), documents, or other objects that have been attached to case 1542.

Content from chats and cases may be brought together in discussions that center around particular cases. As shown in FIG. 15, a discussion interface 1520 presented on a participant device 102 organizes information from a particular chat and a particular case into a single view focused on the case and maintained for users participating in the chat. For example, discussion interface 1520 is directed to discussion 1522, which includes content from both chat 1502 (“Mount Sinai Orthopedics”) and case 1542 (“The mysterious case”). Because discussion 1522 includes content from multiple separately-maintained collections of content, it may be accessed in multiple ways. For example, discussion 1522 may be accessed from either the chat interface 1510 for chat 1502 or the case interface 1530 for case 1542.

In some embodiments, a discussion may be limited to bringing together content from a single chat and a single case. For example, the case interface 1540 for case 1542 shows two different discussions: discussion 1534 and discussion 1522. Each discussion is associated with one and only one chat from the chat listing interface 1500. While both of discussions 1534 and 1522 are associated with case 1542, only one of them is shown at a time on discussion interface 1520 (discussion 1522 in this example), and content from only one chat is accessible for the case 1542 via the discussion interface 1520 (chat 1502 in this example). In other words, chats may be associated with cases on a many-to-many basis. However, each association of chat and case is a one-to-one association maintained in the form of a discussion.

Each discussion may include any number of messages that are separate from the other messages of the chat with which the discussion is associated. For example, participants in discussion 1522 may add and access messages, such as discussion message 1524 via discussion interface 1520. Messages added to discussions may also be accessible via chat interface 1510. For example, discussion 1522 with discussion message 1524 is presented within chat 1502 on chat interface 1510. Thus, participants in a chat are also able to access, in a single location, both chat messages and discussion messages for cases with which the chat is associated.

Presentation of discussion messages with chat messages in the chat interface 1510 may be managed according to a two-level approach in which chat messages are handled and presented differently from discussion messages such that chat threads and discussion threads remain distinct but are organized and presented in a single interface. Advantageously, chat messages and discussion messages may be presented using different display characteristics, enabling the user to distinguish between a message which is part of a discussion and a message which is not associated with a discussion (and thus not related to a medical case). For example, chat messages may be presented with different display characteristics such as in different colors and/or within display objects having a different shape than discussion messages (e.g., oval versus rectangular display objects). In the example of FIG. 15, received and sent chat messages have blue and gray background colors, respectively, while discussion threads and messages have varying shades of green color. In some embodiments, discussion threads within a chat may be collated, presenting only the title/headline/first message of the discussion, while chat messages not associated with a discussion may all be presented in the interface.

In some embodiments, chat messages 1512 may be presented via chat interface 1510 in chronological order (e.g., sorted from earliest message at the top to most recent message at the bottom, permitting a user to scroll up to access earlier chat messages 1512 if not all chat messages fit within chat interface 1510), while for each discussion, only the most recently-added discussion message 1524 is presented within a display object that is representative of the discussion 1522 as a whole. The display object for the discussion 1522 may be presented within the listing of messages at a location corresponding to the time at which the most recently-added discussion message 1524 was added to the discussion 1522. For example, as shown in FIG. 15 discussion message 1524 may have been added to discussion 1522 after the three chat messages 1512 illustrated at the top of chat interface 1510, but before the chat message 1514 illustrated near the bottom chat interface 1510. Moreover, the display object for discussion 1522 may present a preview or limited number of discussion messages of the discussion 1522, even if multiple discussion messages have been added to the discussion after other chat messages 1512 presented in the chat interface 1510. For example, the display object for discussion 1522 may present only a single most recently-added discussion message 1524. User activation of the message 1524 or the display object for the discussion 1522 may cause presentation of discussion interface 1520.

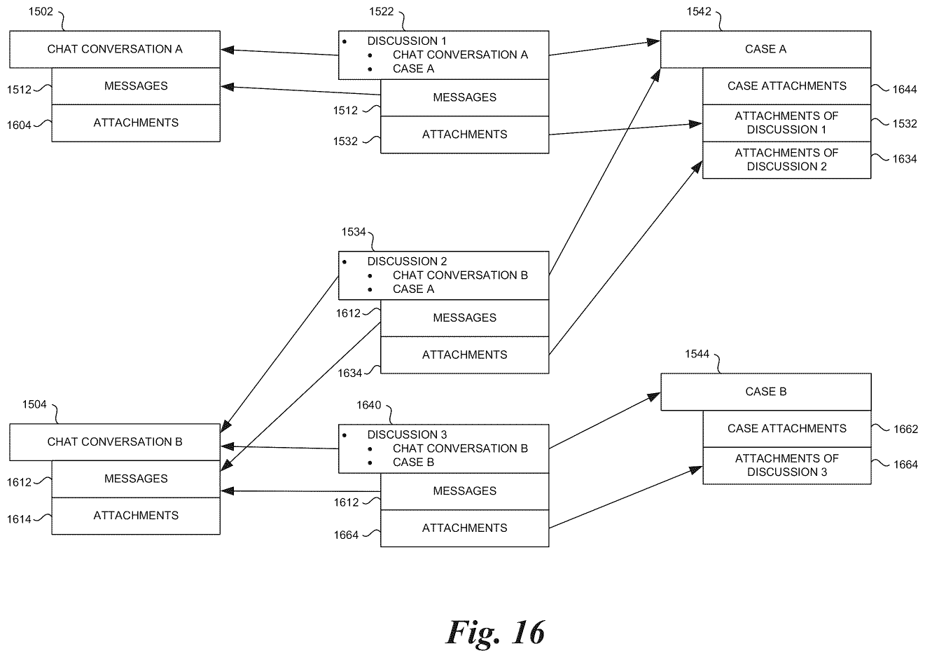

FIG. 16 illustrates an example of how chats and cases may be maintained in a many-to-many association, with each association of chat and case being maintained in the form of a single discussion. As shown, chats 1502 and 1504 each have their own sets of messages and attachments: chat 1502 has chat messages 1512 and attachments 1604, while chat 1504 has messages 1612 and attachments 1614. The messages and attachments of one chat are not accessible within another chat, even if a same user is participating in both chats. Instead, the user accesses each chat separately. Although the chats are each shown with messages and attachments, the examples are provided for purposes of illustration only. In other examples, a particular chat may have only messages or only attachments.

A case may have different types or tiers of attachments. For example, a case may have case attachments that are assigned only to the case, not to any discussion, and therefore are inaccessible outside of a case interface 1530. Thus, case attachments are not accessible via a discussion interface 1520, and may not be accessible to anyone but a case owner or case administrator if access to the case interface 1530 is limited to the case owner or case administrator. As another example, a case may have discussion attachments that are assigned to a particular discussion regarding the case and are accessible via a discussion interface 1520. Thus, the discussion attachments may be accessible to any chat participations that are part of the chat that has a one-to-one association with the case.

As shown in FIG. 16, cases 1542 and 1544 each have their own case attachments: case attachments 1644 and case attachments 1662, respectively. In addition, case 1542 has discussion attachments grouped according to two different discussions: discussion attachments 1532 that are part of discussion 1522, and discussion attachments 1634 that are part of discussion 1534. Case 1544 has discussion attachments 1664 that are part of discussion 1640.

Discussion 1522, by virtue of representing a one-to-one relationship between chat 1502 and case 1542, has chat messages 1512 and discussion attachments 1532 within its discussion content accessible via a discussion interface 1520. As shown in FIG. 15 and described above, a user who is participating in chat 1502 may access, via a discussion interface 1520, chat messages 1512 that are associated with the case 1542. In some embodiments, chat messages 1512 may be maintained in a manner similar to attachments of a case: there may be different types or tiers of messages, including messages that are assigned only to the chat itself and not to any particular case, and messages that assigned to a particular case. Thus, discussion interface 1520 may only provide access to the messages assigned to the corresponding case, and not provide access to the messages assigned to other cases or only to the chat 1502 itself.

Discussion 1534, by virtue of representing a one-to-one relationship between chat 1504 and case 1542, has messages 1612 and discussion attachments 1634 within its discussion content accessible via a discussion interface 1520. Although discussion attachments 1532 for case 1542 are available to participants of chat 1502 as described above, those same attachments are not necessarily available to participants of chat 1504. Discussion 1640, by virtue of representing a one-to-one relationship between chat 1504 and case 1544, has messages 1612 and discussion attachments 1664 within its discussion content accessible via a discussion interface 1520.

### Example Computing Device

FIG. 17 illustrates an example training system computing device 1700 that may be used in some embodiments to provide various features described herein. In some embodiments, the computing device 1700 may include: one or more computer processors 1702, such as physical central processing units (CPUs) or graphics processing units (GPUs); one or more network interfaces 1704, such as a network interface cards (NICs); one or more computer readable medium drives 1706, such as high density disks (HDDs), solid state drives (SDDs), flash drives, and/or other persistent non-transitory computer-readable media; and one or more computer readable memories 1710, such as random access memory (RAM) and/or other volatile non-transitory computer-readable media. The network interface 1704 can provide connectivity to one or more networks or computing devices. The computer processor 1702 can receive information and instructions from other computing devices or services via the network interface 1704. The network interface 1704 can also store data directly to the computer-readable memory 1710. The computer processor 1702 can communicate to and from the computer-readable memory 1710, execute instructions and process data in the computer-readable memory 1710, etc.

The computer-readable memory 1710 may include computer program instructions that the computer processor 1702 executes in order to implement one or more embodiments. The computer-readable memory 1710 can store an operating system 1712 that provides computer program instructions for use by the computer processor 1702 in the general administration and operation of the computing device 1700. The computer-readable memory 1710 can also include capture subsystem instructions 1714 for implementing the features of the capture subsystem 112, control subsystem instructions 1716 for implementing the features of control subsystem 114, conference and chat subsystem instructions 1718 for implementing the features of the conference and chat subsystem 110, other instructions, or any combination thereof.

### Illustrative Embodiments

Some inventive aspects of the disclosure are set forth in the following clauses:

Clause 1. A computer-implemented method for remote control of a live stream video field of view, the computer-implemented method comprising: under control of a handheld computing device comprising a video camera, an output, and one or more processors configured to execute specific computer-executable instructions, obtaining, using the video camera, video data representing a field of view of the video camera; sending a substantially live stream of the video data to a remote device; receiving, from the remote device while continuing to obtain video data representing the field of view, device movement data representing a desired movement of the handheld computing device to change the field of view; and presenting, using the output, a prompt to move the handheld computing device based on the desired movement.

Clause 2. The computer-implemented method of clause 1, wherein receiving the device movement data representing the desired movement comprises receiving data representing a magnitude and direction in which the handheld computing device is to be moved.

Clause 3. The computer-implemented method of any of clauses 1-2, wherein presenting the prompt comprises displaying an arrow indicating a direction in which the handheld computing device is to be moved to satisfy the desired movement.

Clause 4. The computer-implemented method of any of clauses 1-3, further comprising: generating motion data using an inertial motion sensor; determining, based on the motion data, that movement of the handheld computing device has satisfied the desired movement; and ending presentation of the prompt.

Clause 5. The computer-implemented method of any of clauses 1-3, further comprising: analyzing the video data to determine a movement of the handheld computing device; determining that movement of the handheld computing device has satisfied the desired movement; and ending presentation of the prompt.

Clause 6. The computer-implemented method of any of clauses 1-5, further comprising presenting a second prompt in response to determining that movement of the handheld computing device has satisfied the desired movement.

Clause 7. The computer-implemented method of clause 6, wherein presenting the second prompt comprises generating haptic feedback.

Clause 8. The computer-implemented method of clause 6, wherein presenting the second prompt comprises displaying the second prompt.

Clause 9. The computer-implemented method of any of clauses 1-8, further comprising: receiving, from the remote device while continuing to obtain video data representing the field of view, capture setting data representing a capture setting of the video camera to be applied; and applying the capture setting to capture of the video data.

Clause 10. The computer-implemented method of clause 9, wherein applying the capture setting comprises changing one of: an exposure setting, a zoom setting, a color temperature setting, a flash setting, or a focus setting.

Clause 11. The computer-implemented method of clause 10, further comprising: receiving, from the remote device while continuing to obtain video data representing the field of view, second capture setting data representing a second capture setting of the video camera to be applied, wherein the second capture setting is different from the capture setting; and applying the second capture setting to capture of the video data.

Clause 12. The computer-implemented method of any of clauses 1-8, further comprising: receiving, from the remote device while continuing to obtain video data representing the field of view, capture setting data representing a combination of capture settings of the video camera to be applied; and applying the combination of capture settings to capture of the video data.

Clause 13. The computer-implemented method of clause 12, wherein applying the combination of capture settings comprises changing two or more of: an exposure setting, a zoom setting, a color temperature setting, a flash setting, or a focus setting.

Clause 14. The computer-implemented method of any of clauses 1-13, further comprising: analyzing a frame of the video data to determine whether a region in the frame of video data comprises potentially sensitive information; and in response to determining that the region comprises potentially sensitive information, applying a visual mask to the region to generate anonymized video content.

Clause 15. The computer-implemented method of any of clauses 1-14, further comprising: storing at least a portion of the video data in a local data store of the handheld computing device; establishing a bidirectional audio communication connection with the remote device; receiving, from the remote device: playback data representing one or more playback commands for presentation of the video data; and annotation data representing one or more annotations to be presented with the video data; and presenting the portion of the video data from the local data store onto the output with the one or more annotations, wherein the portion of the video data is presented according to the one or more playback commands while maintaining the bidirectional audio communication connection with the remote device.

Clause 16. The computer-implemented method of any of clauses 1-15, further comprising: determining an aspect ratio of a display external to the handheld computing device based at least partly on one or more coordinates associated with the display in the video data; determining an orientation of the display in three-dimensional space at one or more time points in the video data based at least partly on the aspect ratio; and generating a substantially rectangular two-dimensional representation of the display at each of the one or more time points based on the orientation of the display at the one or more time points.

Clause 17. The computer-implemented method of any of clauses 1-15, further comprising: determining an aspect ratio of a display external to the handheld computing device based at least partly on one or more coordinates associated with the display in image data; determining an orientation of the display in three-dimensional space in the image data based at least partly on the aspect ratio; generating a substantially rectangular two-dimensional representation of the display based on the orientation of the display; and generate a cropped, perspective-transformed version of the image data based on the substantially rectangular two-dimensional representation of the display.

Clause 18. A system for remote control of a live stream video field of view, comprising: a camera; a display; and one or more processors programmed by executable instructions to: obtain, using the camera, video data representing a field of view of the camera; send a substantially live stream of the video data to a remote device; receive, from the remote device while continuing to obtain video data representing the field of view, device movement data representing a desired movement of the system to change the field of view; and present, on the display, a prompt to move the system based on the desired movement.

Clause 19. A computer-implemented method for remote control of a live stream video field of view, the computer-implemented method comprising: under control of a handheld computing device comprising a display and one or more processors configured to execute specific computer-executable instructions, receiving, from a remote device, video data representing a field of view of a video camera of the remote device; presenting the video data on the display; generating motion data representing a motion of the handheld computing device during presentation of the video data on the display; determining, based on the motion data, a movement of the handheld computing device; and sending, to the remote device, desired movement data representing a desired movement of the remote device.

Clause 20. The computer-implemented method of clause 19, wherein generating the motion data comprises generating the motion data using an inertial motion sensor of the handheld computing device.

Clause 21. The computer-implemented method of clause 19, wherein generating the motion data comprises generating the motion data based on an analysis of second video data obtained from a video camera of the handheld computing device.

Clause 22. The computer-implemented method of any of clauses 19-21, further comprising receiving user input activating a guidance mode, wherein the motion data is generated in response to activating the guidance mode.

Clause 23. The computer-implemented method of any of clauses 19-22, further comprising: receiving, while continuing to present the video data on the display, user input representing a capture setting of the video camera to be applied; and sending, to the remote device, setting data representing the capture setting to be applied.

Clause 24. The computer-implemented method of clause 23, wherein receiving the user input representing the capture setting comprises receiving input representing a change to at least one of: an exposure setting, a zoom setting, a color temperature setting, a flash setting, or a focus setting.

Clause 25. A system for remote control of a live stream video field of view, comprising: a display; and one or more processors programmed by executable instructions to: receive, from a remote device, video data representing a field of view of a video camera of the remote device; present the video data on the display; generate motion data representing a motion of the system during presentation of the video data on the display; determine, based on the motion data, a movement of the system; and send, to the remote device, desired movement data representing a desired movement of the remote device.

Clause 26. A system comprising: a control device comprising a first display and first processor; and a capture device comprising a camera, a second display, and a second processor; wherein the control device is configured to: receive, from the capture device, video data representing a field of view of the camera; present the video data on the first display; generate motion data representing a motion of the control device during presentation of the video data on the first display; determine, based on the motion data, a movement of the control device; and send, to the capture device, desired movement data representing a desired movement of the capture device; and wherein the capture device is configured to: receive, from the control device while capturing the video data representing the field of view of the camera, device movement data representing a desired movement of the capture device to change the field of view; and present a prompt on the second display to move the capture device based on the desired movement.

Clause 27. The system of clause 26, wherein the device movement data represents a magnitude and direction in which the capture device is to be moved.

Clause 28. The system of any of clauses 26 or 27, wherein the prompt comprises an arrow indicating a direction in which the capture device is to be moved to satisfy the desired movement.

Clause 29. The system of any of clauses 26-28, wherein the capture device comprises an inertial motion sensor configured to generate motion data, and wherein the capture device is further configured to: determine, based on the motion data, that movement of the capture device has satisfied the desired movement; and end presentation of the prompt.

Clause 30. The system of any of clauses 26-28, wherein the capture device is further configured to: analyze the video data to determine a movement of the capture device; determine that movement of the capture device has satisfied the desired movement; and end presentation of the prompt.

Clause 31. The system of any of clauses 26-30, wherein the capture device is further configured to present a second prompt in response to determining that movement of the capture device has satisfied the desired movement.

Clause 32. The system of any of clauses 26-31, wherein the capture device is further configured to: receive, from the control device while continuing to obtain video data representing the field of view, capture setting data representing a capture setting of the camera to be applied; and apply the capture setting to capture of the video data.

Clause 33. The system of clause 32, wherein the capture setting comprises at least one of: an exposure setting, a zoom setting, a color temperature setting, a flash setting, or a focus setting.

Clause 34. The system of any of clauses 26-33, wherein the control device comprises an inertial motion sensor, and wherein the motion data is based on output of the inertial motion sensor.

Clause 35. The system of any of clauses 26-33, wherein the motion data is based on an analysis of second video data obtained from a video camera of the control device.

Clause 36. The system of any of clauses 26-35, wherein the control device is further configured to receive user input activating a guidance mode, wherein the motion data is generated in response to activating the guidance mode.

Clause 37. A computer-implemented method for masking potentially sensitive information in video content, the computer-implemented method comprising: under control of a handheld computing device comprising a video camera and one or more processors configured to execute specific computer-executable instructions, obtaining, using the video camera, video data representing a view of a display external to the handheld computing device during presentation of content on the display, wherein the content comprises one or more regions of text; analyzing a frame of the video data using a machine learning model trained to generate sensitive information classification output representing whether a region of text in the frame of video data comprises sensitive information; determining, based on the sensitive information classification output, to apply a visual mask to at least one region of the one or more regions of text; and applying the visual mask to the at least one region to generate anonymized video content.

Clause 38. The computer-implemented method of clause 37, further comprising determining, based on the sensitive information classification output, not to apply a visual mask to at least a second region of the one or more regions of text.

Clause 39. The computer-implemented method of clause 37, further comprising: receiving user input selecting the visual mask to be removed; and removing the visual mask from the at least one region.

Clause 40. The computer-implemented method of clause 39, further comprising: sending, to a remote computing device, feedback data representing the user input selecting the visual mask to be removed; and receiving, from the remote computing device, an updated machine learning model trained based at least partly on the feedback data.

Clause 41. The computer-implemented method of clause 37, further comprising: receiving user input selecting second region of text to which a second visual mask is to be applied; and applying the second visual mask to the second region of text.

Clause 42. The computer-implemented method of clause 41, further comprising: sending, to a remote computing device, feedback data representing the user input selecting the second region of text; and receiving, from the remote computing device, an updated machine learning model trained based at least partly on the feedback data.

Clause 43. The computer-implemented method of any of clauses 37-42, further comprising: incrementing a frame counter based on the frame; and determining that the frame counter satisfies a processing interval, wherein analyzing the video data is performed in response to determining that the frame counter satisfies the processing interval.

Clause 44. The computer-implemented method of clause 43, further comprising; determining to apply the visual mask to the at least one region in one or more subsequent frames; and applying the visual mask to the at least one region in the one or more subsequent frames without analyzing the one or more subsequent frames using the machine learning model.

Clause 45. The computer-implemented method of clause 43, further comprising; determining to apply the visual mask to the at least one region in one or more prior frames; and applying the visual mask to the at least one region in the one or more prior frames without analyzing the one or more prior frames using the machine learning model.

Clause 46. The computer-implemented method of clause 45, further comprising performing optical character recognition on the frame to detect the at least one region of text.

Clause 47. The computer-implemented method of any of clauses 37-46, further comprising sending the anonymized video content to a remote computing device.

Clause 48. The computer-implemented method of any of clauses 37-47, further comprising storing the video data and the anonymized video content.

Clause 49. A system for masking potentially sensitive information in video content, comprising: a camera; and one or more processors programmed by executable instructions to: obtain, using the camera, video data representing a view of a display external to the system during presentation of content on the display, wherein the content comprises one or more regions of text; analyze a frame of the video data using a machine learning model trained to generate sensitive information classification output representing whether a region of text in the frame of video data comprises sensitive information; determine, based on the sensitive information classification output, to apply a visual mask to at least one region of the one or more regions of text; and apply the visual mask to the at least one region to generate anonymized video content.

Clause 50. A computer-implemented method for masking potentially sensitive information in video content, the computer-implemented method comprising: under control of a handheld computing device comprising a video camera and one or more processors configured to execute specific computer-executable instructions, incrementing a frame counter based on receipt of a frame of video data generated using the video camera, wherein the video data comprises one or more regions of potentially sensitive information; determining that the frame counter satisfies a processing interval; in response to determining that the frame counter satisfies the processing interval, analyzing a frame of the video data using a machine learning model trained to generate sensitive information classification output representing whether a region of potentially sensitive information is present in the frame of video data comprises sensitive information; determining, based on the sensitive information classification output, to apply a visual mask to at least one region of the one or more regions of potentially sensitive information; and applying the visual mask to the at least one region to generate anonymized video content.

Clause 51. The computer-implemented method of clause 50, further comprising; determining to apply the visual mask to the at least one region in one or more subsequent frames; and applying the visual mask to the at least one region in the one or more subsequent frames without analyzing the one or more subsequent frames using the machine learning model.

Clause 52. The computer-implemented method of clause 50, further comprising; determining to apply the visual mask to the at least one region in one or more prior frames; and applying the visual mask to the at least one region in the one or more prior frames without analyzing the one or more prior frames using the machine learning model.

Clause 53. The computer-implemented method of any of clauses 50-52, further comprising determining, based on the sensitive information classification output, not to apply a visual mask to at least a second region of the one or more regions of potentially sensitive information.

Clause 54. The computer-implemented method of any of clauses 50-53, further comprising: receiving user input selecting the visual mask to be removed; and removing the visual mask from the at least one region.

Clause 55. The computer-implemented method of clause 54, further comprising: sending, to a remote computing device, feedback data representing the user input selecting the visual mask to be removed; and receiving, from the remote computing device, an updated machine learning model trained based at least partly on the feedback data.

Clause 56. The computer-implemented method of any of clauses 50-55 further comprising: receiving user input selecting second region of potentially sensitive information to which a second visual mask is to be applied; and applying the second visual mask to the second region of potentially sensitive information.

Clause 57. The computer-implemented method of clause 56, further comprising: sending, to a remote computing device, feedback data representing the user input selecting the second region of potentially sensitive information; and receiving, from the remote computing device, an updated machine learning model trained based at least partly on the feedback data.

Clause 58. The computer-implemented method of any of clauses 50-57, wherein at least one of the one or more regions of potentially sensitive information comprises textual potentially sensitive information.

Clause 59. The computer-implemented method of clause 58, further comprising performing optical character recognition on the frame to detect the textual potentially sensitive information.

Clause 60. The computer-implemented method of any of clauses 50-59, wherein at least one of the one or more regions of potentially sensitive information comprises non-textual potentially sensitive information.

Clause 61. The computer-implemented method of clause 60, wherein the non-textual potentially sensitive information comprises at least one of a face or a facial feature.

Clause 62. The computer-implemented method of clause 61, further comprising using a facial recognition model on the frame to detect the at least one region of non-textual potentially sensitive information.

Clause 63. The computer-implemented method of any of clauses 50-62, further comprising sending the anonymized video content to a remote computing device.

Clause 64. The computer-implemented method of any of clauses 50-63, further comprising storing the video data and the anonymized video content.

Clause 65. A system for masking potentially sensitive information, comprising: a video camera; and one or more processors programmed by executable instructions to: increment a frame counter based on receipt of a frame of video data generated using the video camera, wherein the video data comprises one or more regions of potentially sensitive information; determine that the frame counter satisfies a processing interval; in response to determining that the frame counter satisfies the processing interval, analyze a frame of the video data using a machine learning model trained to generate sensitive information classification output representing whether a region of potentially sensitive information is present in the frame of video data comprises sensitive information; determine, based on the sensitive information classification output, to apply a visual mask to at least one region of the one or more regions of potentially sensitive information; and apply the visual mask to the at least one region to generate anonymized video content.

Clause 66. A computer-implemented method for sharing high-resolution content on demand during communication sessions, the computer-implemented method comprising: under control of a handheld computing device comprising a video camera, a local data store, a display, and one or more processors configured to execute specific computer-executable instructions, establishing a bidirectional audio communication connection with a remote computing device; presenting first video data generated using the video camera, wherein the first video data represents a field of view of the video camera at a first resolution; storing the first video data in the local data store, sending second video data to a remote computing device, wherein the second video data comprises a version of the first video data in a second resolution lower than the first resolution; and sending, while continuing to obtain and present video data generated using the video camera and maintaining the bidirectional audio communication connection with the remote computing device, at least a portion of the first video data to the remote computing device in response to a request for the portion of the first video data.

Clause 67. The computer-implemented method of clause 66, wherein sending at least the portion of the first video data to the remote computing device is performed while continuing to send the second video data to the remote computing device.

Clause 68. The computer-implemented method of clause 66, further comprising obtaining a single frame of the first video data from the local data store based on a frame identifier included in the request for the portion of the first video data, wherein sending at least the portion of the first video data comprises sending the single frame of the first video data.

Clause 69. The computer-implemented method of clause 66, further comprising obtaining a series of frames of the first video data from the local data store based on a time range identifier in the request for the portion of the first video data, wherein sending at least the portion of the first video data comprises sending the series of frames of the first video data.

Clause 70. The computer-implemented method of any of clauses 66-69, further comprising generating the second video data based on a network condition of a network over which the handheld computing device is to send video data to the remote computing device.

Clause 71. The computer-implemented method of any of clauses 66-70, further comprising: receiving, from the remote computing device, interaction data representing a user interaction with a portion of the first video data; loading the portion of the first video data from the local data store; and presenting the portion of the first video data on the display based on the interaction data.

Clause 72. The computer-implemented method of clause 71, wherein receiving the interaction data comprises receiving data representing a playback command to be applied to playback of the portion of the first video data.

Clause 73. The computer-implemented method of clauses 71 or 72, wherein receiving the interaction data comprises receiving data representing an annotation to be applied to presentation of the portion of the first video data.

Clause 74. The computer-implemented method of any of clauses 71-73, wherein presenting the portion of the first video data on the display is performed in substantially real time with presentation of the portion of the video data on the remote computing device.

Clause 75. The computer-implemented method of any of clauses 66-74, further comprising: determining an aspect ratio of a display external to the handheld computing device based at least partly on one or more coordinates associated with the display in the video data; determining an orientation of the display in three-dimensional space at one or more time points in the video data based at least partly on the aspect ratio; and generating a substantially rectangular two-dimensional representation of the display at each of the one or more time points based on the orientation of the display at the one or more time points.

Clause 76. The computer-implemented method of clause 75, further comprising sending, to the remote computing device, third video data comprising a version of the substantially rectangular two-dimensional representation of the display in the second resolution.

Clause 77. The computer-implemented method of any of clauses 66-74, further comprising: determining an aspect ratio of a display external to the handheld computing device based at least partly on one or more coordinates associated with the display in image data; determining an orientation of the display in three-dimensional space in the image data based at least partly on the aspect ratio; generating a substantially rectangular two-dimensional representation of the display based on the orientation of the display; and generate a cropped, perspective-transformed version of the image data based on the substantially rectangular two-dimensional representation of the display.

Clause 78. The computer-implemented method of any of clauses 77, further comprising sending, to the remote computing device, a version of the cropped, perspective-transformed version of the image data in the second resolution.

Clause 79. The computer-implemented method of any of clauses 66-78, further comprising: analyzing a frame of the video data to determine whether a region in the frame of video data comprises potentially sensitive information; and in response to determining that the region comprises potentially sensitive information, applying a visual mask to the region to generate anonymized video content.

Clause 80. The computer-implemented method of any of clauses 66-79, wherein sending at least the portion of the first video data to the remote computing device comprises sending at least the portion of the first video data over a first network connection of a plurality of network connections; and wherein sending the second video data to the remote computing device comprises sending the second video data over a second network connection of the plurality of network connections.

Clause 81. The computer-implemented method of clauses 80, wherein establishing the bidirectional audio communication connection with the remote computing device comprises establishing a third network connection of the plurality of network connections.

Clause 82. The computer-implemented method of any of clauses 66-81, wherein sending the second video data and at least the portion of the first video data to the remote computing device comprises sending the second video data and at least the portion of the first video data to a second handheld computing device.

Clause 83. A system for sharing high-resolution content on demand during communication sessions, comprising: a video camera; a local data store; a display; and one or more processors programmed by executable instructions to: establish a bidirectional audio communication connection with a remote computing device; present first video data generated using the video camera, wherein the first video data represents a field of view of the video camera at a first resolution; store the first video data in the local data store, send second video data to a remote computing device, wherein the second video data comprises a version of the first video data in a second resolution lower than the first resolution; and send, while continuing to obtain and present video data generated using the video camera and maintaining the bidirectional audio communication connection with the remote computing device, at least a portion of the first video data to the remote computing device in response to a request for the portion of the first video data.

Clause 84. A computer-implemented method for cross-device content viewing, the computer-implemented method comprising: under control of a handheld computing device comprising a local data store, a display, and one or more processors configured to execute specific computer-executable instructions, storing video data in the local data store; establishing a bidirectional audio communication connection with a remote computing device; receiving, from the remote computing device, playback data representing a playback command for presentation of the video data; and presenting the video data from the local data store on the display according to the playback command substantially simultaneously with presentation of the video data on the remote computing device according to the playback command, wherein the video data is presented while maintaining the bidirectional audio communication connection with the remote computing device.

Clause 85. The computer-implemented method of clause 84, further comprising storing, by the remote computing device, the video data in a local data store of the remote computing device, wherein presentation of the video data on the remote computing device comprises presenting, by the remote computing device, the video data from the local data store of the remote computing device on a display of the remote computing device.

Clause 86. The computer-implemented method of clause 85, further comprising: sending second playback data representing a second playback command initiated on the handheld computing device to the remote computing device subsequent to receiving the playback command; presenting, by the handheld computing device, the video data from the local data store on the display according to the second playback command; and presenting, by the remote computing device, the video data from the local data store of the remote computing device on the display of the remote computing device according to the second playback command substantially simultaneously with presenting the video data by the handheld computing device according to the second playback command.

Clause 87. The computer-implemented method of any of clauses 84-86, further comprising receiving the video data from the remote computing device.

Clause 88. The computer-implemented method of any of clauses 84-86, further comprising: generating the video data using a video camera; and sending the video data to the remote computing device.

Clause 89. The computer-implemented method of any of clauses 84-88, further comprising receiving, from the remote computing device, annotation data representing one or more annotations to be presented with the video data, wherein presenting the video data comprises presenting the one or more annotations.

Clause 90. The computer-implemented method of any of clauses 84-89, wherein presenting the video data according to the playback command comprises at least one of: initiating playback of the video data, pausing playback of the video data, stopping playback of the video data, rewinding the video data, fast forwarding the video data, applying a degree of zoom to the video data, or presenting an annotation to the video data.

Clause 91. The computer-implemented method of any of clauses 84-90, further comprising: receiving, from a second remote computing device, second playback data representing a second playback command, wherein the second playback data is received during presentation of the video data according to the playback command; and presenting the video data according to the second playback command.

Clause 92. The computer-implemented method of clause 91, wherein presenting the video data according to the second playback command comprises altering presentation of the video data being presented according to the playback command.

Clause 93. The computer-implemented method of any of clauses 84-90, further comprising: receiving, from a second remote computing device, second playback data representing a second playback command, wherein the second playback data is received during presentation of the video data according to the playback command; and determining not to present the video data according to the second playback command based on at least one of the remote computing device being or the playback command being associated with a higher level of a control hierarchy than at least one of the second remote computing device or the second playback command.

Clause 94. The computer-implemented method of any of clauses 84-90, further comprising: detecting a user input on the handheld computing device; determining that the user input represents a second playback command, wherein the user input occurs subsequent to receiving the playback data and during presentation of the video data according to the playback command; and presenting the video data according to the second playback command.

Clause 95. The computer-implemented method of clause 94, wherein presenting the video data according to the second playback command comprises altering presentation of the video data being presented according to the playback command.

Clause 96. The computer-implemented method of any of clauses 94 or 95, further comprising sending second playback data representing the second playback command to the remote computing device, wherein the remote computing device presents the video data according to the second playback command substantially simultaneously with presentation of the video data on the handheld computing device according to the second playback command.

Clause 97. The computer-implemented method of any of clauses 84-90, further comprising: detecting a user input on the handheld computing device; determining that the user input represents a second playback command; and determining not to present the video data according to the second playback command based on at least one of the remote computing device or the playback command being associated with a higher level of a control hierarchy than at least one of the handheld computing device or the second playback command.

Clause 98. The computer-implemented method of clause 97, further comprising sending second playback data representing the second playback command to the remote computing device, wherein the remote computing device determines not to present the video data according to the second playback command.

Clause 99. A system for cross-device content viewing, comprising: a local data store; a display; and one or more processors programmed by executable instructions to: store video data in the local data store; establish a bidirectional audio communication connection with a remote computing device; receive, from the remote computing device, playback data representing a playback command for presentation of the video data; and present the video data from the local data store on the display according to the playback command substantially simultaneously with presentation of the video data on the remote computing device according to the playback command, wherein the video data is presented while maintaining the bidirectional audio communication connection with the remote computing device.

Clause 100. A system for cross-device content viewing, comprising: a plurality of computing devices, wherein each computing device of the plurality of computing devices comprises a local data store, a display, and a processor programmed by executable instructions, wherein: each computing device of the plurality of computing devices presents a same video data from a respective local data store substantially simultaneously with each other computing device of the plurality of computing devices, wherein the video data is presented according to a first playback command from a computing device of the plurality of computing devices, and wherein at least one of the computing device or the first playback command is associated with a first level of a control hierarchy; and each computing device of the plurality of computing devices determines not to apply a second playback command to presentation of the video data based on a second level of the control hierarchy with which at least one of the second playback command or a source of the second playback command is associated.

Clause 101. The system of clause 100, wherein the source of the second playback command is the computing device.

Clause 102. The system of clause 100, wherein the source of the second playback command is a second computing device of the plurality of computing devices.

Clause 103. The system of any of clauses 100-102, wherein the second playback command is issued subsequent to the first playback command.

Clause 104. The system of any of clauses 100-103, wherein the first level of the control hierarchy takes precedence over the second level.

Clause 105. A computer-implemented method for cross-device content viewing, comprising: presenting, by each computing device of a plurality of computing devices, a same video data from a respective local data store of each computing device substantially simultaneously with each other computing device of the plurality of computing devices, wherein the video data is presented according to a first playback command from a computing device of the plurality of computing devices, and wherein at least one of the computing device or the first playback command is associated with a first level of a control hierarchy; and determining, by each computing device of the plurality of computing devices, not to apply a second playback command to presentation of the video data based on a second level of the control hierarchy with which at least one of the second playback command or a source of the second playback command is associated.

Clause 106. The computer-implemented method of clause 105, wherein presenting the video data according to the playback command comprises at least one of: initiating playback of the video data, pausing playback of the video data, stopping playback of the video data, rewinding the video data, fast forwarding the video data, applying a degree of zoom to the video data, or presenting an annotation to the video data.

Clause 107. A computer-implemented method for obtaining high-resolution content on demand during communication sessions, the computer-implemented method comprising: under control of a handheld computing device comprising a display, a local data store, and one or more processors configured to execute specific computer-executable instructions, establishing a bidirectional audio communication connection with a remote computing device; presenting a first version of video data received from the remote computing device, wherein the video data represents a field of view of a video camera of the remote computing device at a first resolution; sending a request to the remote computing device for a second version of at least a portion of the video data in a second resolution higher than the first resolution; storing the second version in the local data store upon receipt from the remote computing device; and presenting the second version from the local data store while maintaining the bidirectional audio communication connection with the remote computing device.

Clause 108. The computer-implemented method of clause 107, further comprising: determining a frame identifier of a single frame of the video data, wherein the request includes the frame identifier; and receiving the single frame of the video data from the remote computing device in response to the request.

Clause 109. The computer-implemented method of clause 107, further comprising: determining a time range identifier of a series of frames of the video data, wherein the request includes the time range identifier; and receiving the series of frames of the video data from the remote computing device in response to the request.

Clause 110. The computer-implemented method of any of clauses 107-109, further comprising: generating interaction data representing a user interaction with a portion of the second version of video data; adjusting presentation of the portion of the second version of video data on the display based on the interaction data; and sending the interaction data to the remote computing device.

Clause 111. The computer-implemented method of clause 110, wherein generating the interaction data comprises generating data representing a playback command to be applied to playback of the portion of the second version of video data.

Clause 112. The computer-implemented method of clause 110, wherein generating the interaction data comprises generating data representing an annotation to be applied to presentation of the portion of the second version of video data.

Clause 113. A system for obtaining high-resolution content on demand during communication sessions, comprising: a display; a local data store; and one or more processors programmed by executable instructions to: establish a bidirectional audio communication connection with a remote computing device; present a first version of video data received from the remote computing device, wherein the video data represents a field of view of a video camera of the remote computing device at a first resolution; send a request to the remote computing device for a second version of at least a portion of the video data in a second resolution higher than the first resolution; store the second version in the local data store upon receipt from the remote computing device; and present the second version from the local data store while maintaining the bidirectional audio communication connection with the remote computing device.

Clause 114. A computer-implemented method for generating non-destructive annotated content, the computer-implemented method comprising: under control of a computing device comprising a local data store, a display, and one or more processors configured to execute specific computer-executable instructions, presenting one or more content items from the local data store on the display; receiving input data representing one or more modifications to be made to a presentation of at least a first content item of the one or more content items; generating an annotation file comprising annotation metadata specifying a timeline for presentation of the one or more content items and the one or more modifications, wherein the annotation file is separate from the one or more content items; and sending, to a remote computing device, the annotation file and the one or more content items.

Clause 115. The computer-implemented method of clause 114, further comprising: generating an audio recording; including, in the annotation file, second annotation metadata regarding presentation of the audio recording according to the timeline; and sending the audio recording to the remote computing device.

Clause 116. The computer-implemented method of any of clauses 114 or 115, wherein presenting the one or more content items comprises presenting at least one image and at least one video.

Clause 117. The computer-implemented method of any of clauses 114 or 115, wherein presenting the one or more content items comprises presenting at least one image or at least one video.

Clause 118. The computer-implemented method of any of clauses 114 or 115, wherein presenting the one or more content items comprises presenting a plurality of videos.

Clause 119. The computer-implemented method of any of clauses 114 or 115, wherein presenting the one or more content items comprises presenting a plurality of images.

Clause 120. The computer-implemented method of any of clauses 114-119, wherein receiving the input data representing the one or more modifications comprises receiving input data representing a drawing overlay to be presented with the first content item.

Clause 121. The computer-implemented method of clause 120, wherein generating the annotation file comprises generating at least a portion of the annotation metadata as instructions for presenting a vector graphic corresponding to the drawing overlay.

Clause 122. The computer-implemented method of any of clauses 114-121, wherein receiving the input data representing the one or more modifications comprises receiving input data representing a text overlay to be presented with the first content item; and wherein generating the annotation file comprises generating at least a portion of the annotation metadata as instructions for presenting the text overlay with the first content item.

Clause 123. The computer-implemented method of any of clauses 114-122, wherein receiving the input data representing the one or more modifications comprises receiving input data representing a cursor movement to be presented with the first content item; and wherein generating the annotation file comprises generating at least a portion of the annotation metadata as instructions for presenting the cursor movement.

Clause 124. The computer-implemented method of any of clauses 114-123, wherein receiving the input data representing the one or more modifications comprises receiving input data representing a playback command for presenting the first content item; and wherein generating the annotation file comprises generating at least a portion of the annotation metadata as instructions for executing the playback command.

Clause 125. The computer-implemented method of any of clauses 114-124, further comprising: generating an augmentation file specifying one or more augmentations to be made to a raw content item corresponding to a content item of the one or more content items, wherein the augmentation file is separate from the raw content item.

Clause 126. The computer-implemented method of clause 125, wherein generating the augmentation file comprises determining one or more regions of potentially sensitive information to be masked.

Clause 127. The computer-implemented method of clause 125, wherein generating the augmentation file comprises determining cropping and stabilization to be applied to the raw content item.

Clause 128. A system for generating non-destructive annotated content, comprising: a local data store; a display; and one or more processors programmed by executable instructions to: present one or more content items from the local data store on the display; receive input data representing one or more modifications to be made to a presentation of at least a first content item of the one or more content items; generate an annotation file comprising annotation metadata specifying a timeline for presentation of the one or more content items and the one or more modifications, wherein the annotation file is separate from the one or more content items; and send, to a remote computing device, the annotation file and the one or more content items.

Clause 129. A computer-implemented method for presenting non-destructive annotated content, the computer-implemented method comprising: under control of a computing device comprising a local data store, a display, and one or more processors configured to execute specific computer-executable instructions, receiving, from a remote computing device, an annotation file and one or more content items, wherein the annotation file is separate from the one or more content items, and wherein the annotation file comprises annotation metadata specifying a timeline for one or more modifications to presentation of the one or more content items; presenting the one or more content items with the one or more modifications based on the annotation file; and presenting at least one content item of the one or more content items without any modification specified by the annotation file.

Clause 130. The computer-implemented method of clause 129, wherein presenting the one or more content items comprises presenting at least one image and at least one video.

Clause 131. The computer-implemented method of clause 129, wherein presenting the one or more content items comprises presenting at least one image or at least one video.

Clause 132. The computer-implemented method of clause 129, wherein presenting the one or more content items comprises presenting a plurality of videos.

Clause 133. The computer-implemented method of clause 129, wherein presenting the one or more content items comprises presenting a plurality of images.

Clause 134. The computer-implemented method of any of clauses 129-133, further comprising: receiving input data representing a second set of one or more modifications to be made to a presentation of at least a first content item of the one or more content items; generating a second annotation file comprising second annotation metadata specifying a second timeline for presentation of the first content item and the second set of one or more modifications, wherein the second annotation file is separate from the first content item; and sending, to the remote computing device, the second annotation file.

Clause 135. A system for presenting non-destructive annotated content, comprising: a local data store; a display; and one or more processors programmed by executable instructions to: receive, from a remote computing device, an annotation file and one or more content items, wherein the annotation file is separate from the one or more content items, and wherein the annotation file comprises annotation metadata specifying a timeline for one or more modifications to presentation of the one or more content items; present the one or more content items with the one or more modifications based on the annotation file; and present at least one content item of the one or more content items without any modification specified by the annotation file.

Clause 136. A computer-implemented method for generating video-based views of displays, the computer-implemented method comprising: under control of a handheld computing device comprising a video camera and one or more processors configured to execute specific computer-executable instructions, obtaining, using the video camera, video data representing a view of a display external to the handheld computing device during presentation of content on the display; determining an orientation of the display in three-dimensional space at one or more time points in the video data; and generating a substantially rectangular two-dimensional representation of the display at each of the one or more time points based on the orientation of the display at the one or more time points.

Clause 137. The computer-implemented method of clause 136, further comprising determining an aspect ratio of the display based at least partly on one or more coordinates associated with the display in the video data, wherein determining the orientation of the display is based at least partly on the aspect ratio.

Clause 138. The computer-implemented method of clause 137, wherein determining the aspect ratio comprises identifying one of a plurality of known aspect ratios.

Clause 139. The computer-implemented method of any of clauses 137 or 138, further comprising determining a set of raw frame coordinates representing vertices of an object in the video data.

Clause 140. The computer-implemented method of clause 139, wherein determining the aspect ratio comprises: constructing a set of three-dimensional coordinates for a reference object with a flat surface having a ratio of width to height that is equal to a known aspect ratio; finding a three-dimensional pose of the reference object, wherein the three-dimensional pose is defined in terms of rotation and translation vectors; projecting an object in the video data into two dimensions using the rotation and translation vectors to determine projected coordinates; and measuring a compound distance between the set of raw frame coordinates and the projected coordinates.

Clause 141. The computer-implemented method of any of clauses 136-140, wherein obtaining the video data comprises obtaining video data representing a view of a substantially stationary display while the handheld computing device is moving.

Clause 142. The computer-implemented method of any of clauses 136-141, further comprising generating transformed video data comprising the substantially rectangular two-dimensional representation of the display.

Clause 143. The computer-implemented method of clause 142, further comprising sending the transformed video data to a remote computing device.

Clause 144. The computer-implemented method of any of clauses 136-143, further comprising applying one or more capture settings to be applied based on a presence of the display in the video data.

Clause 145. The computer-implemented method of any of clauses 136-144, further comprising: analyzing a frame of the video data to determine whether a region in the frame of video data comprises potentially sensitive information; and in response to determining that the region comprises potentially sensitive information, applying a visual mask to the region to generate anonymized video content.

Clause 146. A system for generating video-based views of displays, comprising: a video camera; and one or more processors programmed by executable instructions to: obtain, using the video camera, video data representing a view of a display external to the system during presentation of content on the display; determine an orientation of the display in three-dimensional space at one or more time points in the video data; and generate a substantially rectangular two-dimensional representation of the display at each of the one or more time points based on the orientation of the display at the one or more time points.

Clause 147. A computer-implemented method for generating transformed views of generally rectangular objects, the computer-implemented method comprising: under control of a handheld computing device comprising a camera and one or more processors configured to execute specific computer-executable instructions, obtaining, using the camera, input data representing a view of a generally rectangular object external to the handheld computing device; determining an orientation of the generally rectangular object in three-dimensional space; and generating a substantially rectangular two-dimensional representation of the generally rectangular object based on the orientation of the generally rectangular object.

Clause 148. The computer-implemented method of any of clauses 147, further comprising determining an aspect ratio of the generally rectangular object based at least partly on one or more coordinates associated with the generally rectangular object in the input data, wherein determining the orientation of the generally rectangular object is based at least partly on the aspect ratio.

Clause 149. The computer-implemented method of any of clauses 147 or 148, wherein obtaining the input data comprises obtaining one of video data or image data of a printed document.

Clause 150. The computer-implemented method of any of clauses 147 or 148, wherein obtaining the input data comprises obtaining one of video data or image data of a printed image.

Clause 151. The computer-implemented method of any of clauses 147 or 148, wherein obtaining the input data comprises obtaining one of video data or image data of a display screen.

Clause 152. The computer-implemented method of any of clauses 147-151, further comprising generating transformed output data comprising the substantially rectangular two-dimensional representation of the generally rectangular object.

Clause 153. The computer-implemented method of clause 152, further comprising sending the transformed output data to a remote computing device.

Clause 154. A system for generating transformed views of generally rectangular objects, comprising: a camera; and one or more processors programmed by executable instructions to: obtain, using the camera, input data representing a view of a generally rectangular object external to the system; determine an orientation of the generally rectangular object in three-dimensional space; and generate a substantially rectangular two-dimensional representation of the generally rectangular object based on the orientation of the generally rectangular object.

Clause 155. A computer-implemented method for managing messages, the computer-implemented method comprising: under control of a computing device comprising one or more processors configured to execute specific computer-executable instructions, receiving a plurality of messages, wherein individual messages of the plurality of messages are associated with no more than one top-level tier of each of a participant-based hierarchy and a case-based hierarchy; defining a chat conversation comprising a first subset of the plurality of messages, wherein each message of the first subset is associated with a single participant group corresponding to a first top-level tier of the participant-based hierarchy; defining a case discussion comprising a second subset of the plurality of messages, wherein each message of the second subset is associated with the first top-level tier of the participant-based hierarchy and a second top-level tier of the case-based hierarchy; and providing a user interface configured to present, to a user associated with the first top-level tier of the participant-based hierarchy, access to both the first subset and the second subset.

Clause 156. The computer-implemented method of clause 155, further comprising presenting, in the user interface, a user interface control for accessing the second subset.

Clause 157. The computer-implemented method of clause 156, further comprising presenting, in the user interface, a chat message thread comprising at least a portion of the first subset sorted based on a time of creation of each message of the first subset.

Clause 158. The computer-implemented method of clause 157, further comprising determining a location in the chat message thread at which to insert the user interface control based on a time of creation of a most recent message of the second subset.

Clause 159. The computer-implemented method of clause 158, further comprising presenting, in the user interface control, at least a portion of the most recent message of the second subset.

Clause 160. The computer-implemented method of any of clauses 155-159, further comprising providing a second user interface configured to present the second subset and one or more attachments associated with the second top-level tier of the case-based hierarchy, wherein the second user interface excludes at least a portion of the first subset.

Clause 161. The computer-implemented method of any of clauses 155-160, further comprising providing an additional user interface configured to present: one or more attachments associated with the second top-level tier of the case-based hierarchy; a first user interface control to access the case discussion; and a second user interface control to access a second case discussion associated with the second top-level tier of the case-based hierarchy and a different top-level of the participant-based hierarchy than the case discussion.

Clause 162. The computer-implemented method of any of clauses 155-161, further comprising defining a second case discussion comprising a third subset of the plurality of messages, wherein each message of the third subset is associated with a second top-level tier of the participant-based hierarchy and the second top-level tier of the case-based hierarchy.

Clause 163. The computer-implemented method of any of clauses 155-161, further comprising: defining a second chat conversation comprising a third subset of the plurality of messages, wherein each message of the third subset is associated with a single participant group corresponding to a different top-level tier of the participant-based hierarchy than the first top-level tier; and providing a user interface configured to present, to users associated with the first top-level tier of the participant-based hierarchy, access to both the first subset and the second subset.

Clause 164. A system for managing messages, comprising: computer-readable memory storing executable instructions; and one or more processors programmed by the executable instructions to: receive a plurality of messages, wherein individual messages of the plurality of messages are associated with no more than one top-level tier of each of a participant-based hierarchy and a case-based hierarchy; define a chat conversation comprising a first subset of the plurality of messages, wherein each message of the first subset is associated with a single participant group corresponding to a first top-level tier of the participant-based hierarchy; define a case discussion comprising a second subset of the plurality of messages, wherein each message of the second subset is associated with the first top-level tier of the participant-based hierarchy and a second top-level tier of the case-based hierarchy; and provide a user interface configured to present, to a user associated with the first top-level tier of the participant-based hierarchy, access to both the first subset and the second subset.

### Terminology

All of the methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions, or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid-state memory chips or magnetic disks, into a different state. In some embodiments, the computer system may be a cloud-based computing system whose processing resources are shared by multiple distinct business entities or other users.

Depending on the embodiment, certain acts, events, or functions of any of the processes or algorithms described herein can be performed in a different sequence, can be added, merged, or left out altogether (e.g., not all described operations or events are necessary for the practice of the algorithm). Moreover, in certain embodiments, operations or events can be performed concurrently, e.g., through multi-threaded processing, interrupt processing, or multiple processors or processor cores or on other parallel architectures, rather than sequentially.

The various illustrative logical blocks, modules, routines, and algorithm steps described in connection with the embodiments disclosed herein can be implemented as electronic hardware, or combinations of electronic hardware and computer software. To clearly illustrate this interchangeability, various illustrative components, blocks, modules, and steps have been described above generally in terms of their functionality. Whether such functionality is implemented as hardware, or as software that runs on hardware, depends upon the particular application and design conditions imposed on the overall system. The described functionality can be implemented in varying ways for each particular application, but such implementation decisions should not be interpreted as causing a departure from the scope of the disclosure.

Moreover, the various illustrative logical blocks and modules described in connection with the embodiments disclosed herein can be implemented or performed by a machine, such as a processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor device can be a microprocessor, but in the alternative, the processor device can be a controller, microcontroller, or state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without other input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

The following is claimed:

1. A computer-implemented method for remote control of a live stream video field of view, the computer-implemented method comprising:
under control of a handheld computing device comprising a video camera, an output, and one or more processors configured to execute specific computer-executable instructions,
obtaining, using the video camera, video data representing a field of view of the video camera;
sending a substantially live stream of the video data to a remote device;
receiving, from the remote device while continuing to obtain video data representing the field of view, device movement data representing a desired movement of the handheld computing device to change the field of view; and
presenting, using the output, a prompt to move the handheld computing device based on the desired movement.

2. The computer-implemented method of claim 1, wherein receiving the device movement data representing the desired movement comprises receiving data representing a magnitude and direction in which the handheld computing device is to be moved.

3. The computer-implemented method of claim 1, wherein presenting the prompt comprises displaying an arrow indicating a direction in which the handheld computing device is to be moved to satisfy the desired movement.

4. The computer-implemented method of claim 1, further comprising:
generating motion data using an inertial motion sensor;
determining, based on the motion data, that movement of the handheld computing device has satisfied the desired movement; and
ending presentation of the prompt.

5. The computer-implemented method of claim 1, further comprising:
analyzing the video data to determine a movement of the handheld computing device;
determining that movement of the handheld computing device has satisfied the desired movement; and
ending presentation of the prompt.

6. The computer-implemented method of claim 1, further comprising presenting a second prompt in response to determining that movement of the handheld computing device has satisfied the desired movement.

7. The computer-implemented method of claim 6, wherein presenting the second prompt comprises generating haptic feedback.

8. The computer-implemented method of claim 6, wherein presenting the second prompt comprises displaying the second prompt.

9. The computer-implemented method of claim 1, further comprising:
receiving, from the remote device while continuing to obtain video data representing the field of view, capture setting data representing a capture setting of the video camera to be applied; and
applying the capture setting to capture of the video data.

10. The computer-implemented method of claim 9, wherein applying the capture setting comprises changing one of: an exposure setting, a zoom setting, a color temperature setting, a flash setting, or a focus setting.

11. The computer-implemented method of claim 10, further comprising:
receiving, from the remote device while continuing to obtain video data representing the field of view, second capture setting data representing a second capture setting of the video camera to be applied, wherein the second capture setting is different from the capture setting; and
applying the second capture setting to capture of the video data.

12. The computer-implemented method of claim 1, further comprising:
receiving, from the remote device while continuing to obtain video data representing the field of view, capture setting data representing a combination of capture settings of the video camera to be applied; and
applying the combination of capture settings to capture of the video data.

13. The computer-implemented method of claim 12, wherein applying the combination of capture settings comprises changing two or more of: an exposure setting, a zoom setting, a color temperature setting, a flash setting, or a focus setting.

14. The computer-implemented method of claim 1, further comprising:
analyzing a frame of the video data to determine whether a region in the frame of video data comprises potentially sensitive information; and
in response to determining that the region comprises potentially sensitive information, applying a visual mask to the region to generate anonymized video content.

15. The computer-implemented method of claim 1, further comprising:
storing at least a portion of the video data in a local data store of the handheld computing device;
establishing a bidirectional audio communication connection with the remote device;
receiving, from the remote device:
playback data representing one or more playback commands for presentation of the video data; and
annotation data representing one or more annotations to be presented with the video data; and
presenting the portion of the video data from the local data store onto the output with the one or more annotations, wherein the portion of the video data is presented according to the one or more playback commands while maintaining the bidirectional audio communication connection with the remote device.

16. The computer-implemented method of claim 1, further comprising:
determining an aspect ratio of a display external to the handheld computing device based at least partly on one or more coordinates associated with the display in the video data;
determining an orientation of the display in three-dimensional space at one or more time points in the video data based at least partly on the aspect ratio; and
generating a substantially rectangular two-dimensional representation of the display at each of the one or more time points based on the orientation of the display at the one or more time points.

17. The computer-implemented method of claim 1, further comprising:
determining an aspect ratio of a display external to the handheld computing device based at least partly on one or more coordinates associated with the display in image data;
determining an orientation of the display in three-dimensional space in the image data based at least partly on the aspect ratio;
generating a substantially rectangular two-dimensional representation of the display based on the orientation of the display; and
generate a cropped, perspective-transformed version of the image data based on the substantially rectangular two-dimensional representation of the display.

18. A computer-implemented method for remote control of a live stream video field of view, the computer-implemented method comprising:
under control of a handheld computing device comprising a display and one or more processors configured to execute specific computer-executable instructions,
receiving, from a remote device, video data representing a field of view of a video camera of the remote device;
presenting the video data on the display;
generating motion data representing a motion of the handheld computing device during presentation of the video data on the display;
determining, based on the motion data, a movement of the handheld computing device; and
sending, to the remote device, desired movement data representing a desired movement of the remote device.

19. The computer-implemented method of claim 18, wherein generating the motion data comprises generating the motion data using an inertial motion sensor of the handheld computing device.

20. The computer-implemented method of claim 18, wherein generating the motion data comprises generating the motion data based on an analysis of second video data obtained from a video camera of the handheld computing device.
